(19) United States
(12) Patent Application Publication
(10) Pub. No.: US 2009/0258042 A1
(43) Pub. Date: Oct. 15, 2009

(54) Encapsulated Active Materials Containing Adjunct Crosslinkers

(76) Inventors: Theodore James Anastasiou, Holmdel, NJ (US); Johan Gerwin Lodewijk, Middletown, NJ (US); Jeffrey James McElwee, Toms River, NJ (US)

Correspondence Address: INTERNATIONAL FLAVORS & FRAGRANCES INC., 521 WEST 57TH ST, NEW YORK, NY, 10019, US

(21) Appl. No.: 12/102,355

(22) Filed: Apr. 14, 2008

(51) Int. Cl. A61K 8/11; A01N 25/28; A01P 3/00; A01P 1/00; A01P 17/00; A61Q 5/00; A61Q 9/02; A61Q 11/00; A61Q 15/00; A61Q 17/04; C11D 3/50

(52) U.S. Cl. 424/401; 424/497; 424/408; 424/59; 424/70.11; 424/76.1; 424/49; 424/65; 424/73; 512/4; 510/119; 510/130; 510/276; 510/515; 510/220; 252/601; 252/8.81; 252/79.1; 426/89

(57) ABSTRACT

It is an object of the present invention to provide a microcapsule product comprising an active material; said active material encapsulated by a polymeric material to provide a polymer encapsulated material wherein said polymeric material comprises an adjunct crosslinker.

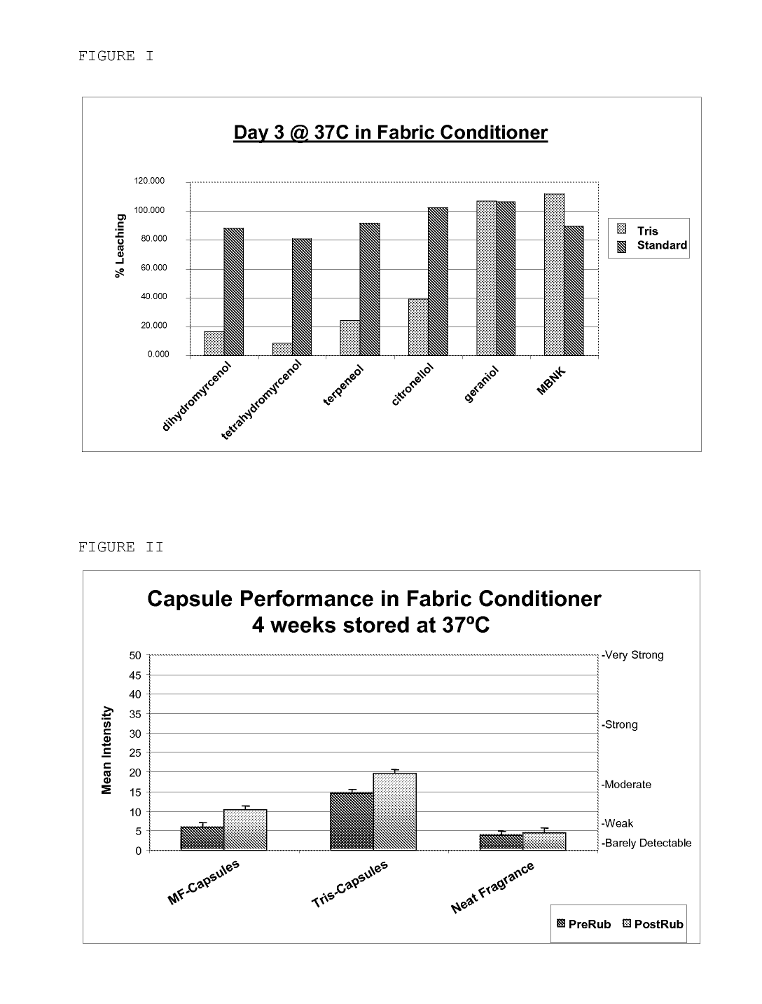

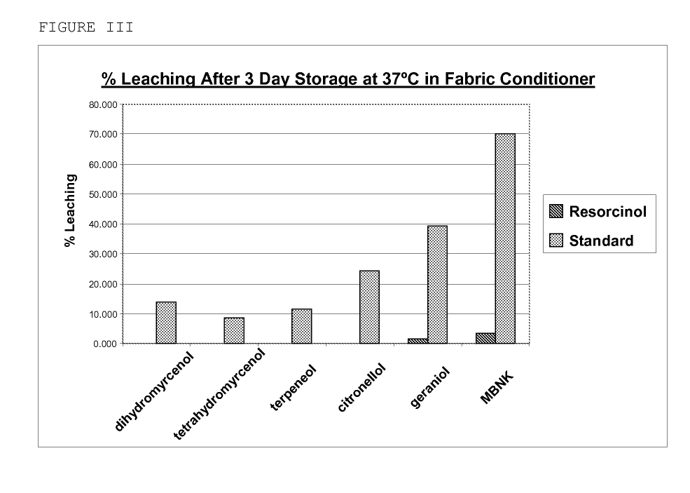

### FIELD OF THE INVENTION

The present invention relates to active materials that are encapsulated with a polymeric material that exhibit reduced formaldehyde levels. The encapsulated fragrance materials are well suited for rinse-off applications associated with personal care and cleaning products.

### BACKGROUND OF THE INVENTION

Fragrance chemicals are used in numerous products to enhance the consumer's enjoyment of a product. Fragrance chemicals are added to consumer products such as laundry detergents, fabric softeners, soaps, detergents, personal care products, such as shampoos, body washes, deodorants and the like, as well as numerous other products.

In order to enhance the effectiveness of the fragrance materials for the user, various technologies have been employed to enhance the delivery of the fragrance materials at the desired time. One widely used technology is encapsulation of the fragrance material in a protective coating. Frequently the protective coating is a polymeric material. The polymeric material is used to protect the fragrance material from evaporation, reaction, oxidation or otherwise dissipating prior to use. A brief overview of polymeric encapsulated fragrance materials is disclosed in the following U.S. patents: U.S. Pat. No. 4,081,384 discloses a softener or anti-stat core coated by a polycondensate suitable for use in a fabric conditioner; U.S. Pat. No. 5,112,688 discloses selected fragrance materials having the proper volatility to be coated by coacervation with micro particles in a wall that can be activated for use in fabric conditioning; U.S. Pat. No. 5,145,842 discloses a solid core of a fatty alcohol, ester, or other solid plus a fragrance coated by an aminoplast shell; and U.S. Pat. No. 6,248,703 discloses various agents including fragrance in an aminoplast shell that is included in an extruded bar soap. The above U.S. patents are hereby incorporated by reference as if set forth in their entirety.

Fragrance microcapsule slurries consist of a fragrance core surrounded by a crosslinked polymeric wall, dispersed in an aqueous medium. The wall often is made up of natural or synthetically derived homopolymers or copolymers containing amide, amine, carboxyl, hydroxyl, thiol and mercaptan functional groups. These polymers are crosslinked with aminoplast type crosslinkers. These crosslinkers are based on melamine-formaldehyde, urea-formaldehyde, glycouril-formaldehyde, benzoguanamine-formaldehyde, ethyleneurea-formaldehyde, dihydroxyethyleneurea-formaldehyde, and hydroxyl (alkoxy) alkyleneurea type chemistries. A byproduct of the crosslinking reaction is formaldehyde, which remains dissolved in the slurry medium (water). The slurry is used “as is” without any attempt to purify it. Thus, the formaldehyde produced in the reaction contributes to the formaldehyde level of the slurry. In addition, formaldehyde is used in the manufacturing process of the crosslinkers which also typically do not undergo any purification. Thus this level also contributes to the final levels of the slurry.

The melamine-formaldehyde crosslinker typically used (Cymel 385) forms a highly crosslinked capsule wall whose permeability decreases with increasing crosslinker levels. However increasing the melamine-formaldehyde crosslinker level also adds more free formaldehyde, potential formaldehyde, and potential melamine to the system. This is undesirable from a safety and regulatory standpoint as well as a public relations standpoint. The excess formaldehyde can be reduced by adding a scavenger, for example.

This method is not preferred however because the scavenger can impact the performance and aesthetics of the capsules, and may create new safety and regulatory concerns over its presence and adducts the scavenger forms with formaldehyde. Furthermore while melamine levels are not a major concern it is preferred that the levels be kept to a minimum.

Up until now the only way to minimize the formaldehyde and melamine levels was to reduce the crosslinker level, however this leads to increased permeability of the capsule wall and thus increased leakage and poor performance.

### SUMMARY OF THE INVENTION

It is an object of the present invention to provide a microcapsule product comprising an active material; said active material encapsulated by a polymeric material to provide a polymer encapsulated material wherein said polymeric material comprises an adjunct crosslinker.

A microcapsule product comprising an active material; said active material encapsulated by a polymeric material to provide a polymer encapsulated material wherein said polymeric material comprises an adjunct crosslinker represented by the following formula:

(R¹-)mX¹(—R²—Y)n (I)

and

(R¹-)m(-R²—Y)nX¹—R³—X²(—R-Z)o(-R⁵)p (II)

where:
X¹ and X² can be equal to C, N or NH, Phosphate, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate. They can be different in structure.
Y and Z can be selected from amine, amide, carboxyl, enolizable carbonyl, hydroxyl, thiol moieties, and mixture thereof.
R¹ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R² can either be equal to zero or equal to CH2, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R³ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R⁴ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R⁵ can either not be present or equal to, CH2, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.

In structure (I) n is larger than 1 but can be equal or less than the maximum substitution possible on the X group. In addition, m+n is equal or less than the maximum substitution possible on the X group. Finally, the values for m and n are integers or non-integers. Non integer values can arise when distributions of substitution are present.

For instance, if X═C, then n can vary between 2 and 4. If X is a benzene ring, then n can vary between 2 and 6.

In structure (II) the values of n and o are at least 1 but can be equal or less than the maximum substitution possible on the X¹ and X² groups, respectively. In addition, m+n is equal or less than the maximum substitution possible on the X1 group minus 1. Furthermore, o+p is equal or less than the maximum substitution possible on the X² group minus 1. Finally, the values for m, n, o and p are integers or non-integers. Non integer values can arise when distributions of substitution are present. For instance is X¹ and X² are carbon atoms m+n< or =3 and o+p< or =3. If X¹ and X² are nitrogen atoms m+n< or =2 and o+p< or =2.

In another embodiment the adjunct crosslinkers can be used in combination with formaldehyde scavengers.

### BRIEF DESCRIPTION OF THE DRAWINGS

FIGURE I: The leaching of tris capsules versus standard capsules is shown below.

FIGURE II: The tris-based capsules leak significantly less than the standard capsules. The sensory performance of tris based capsules versus standard capsules is shown below.

FIGURE III: The leaching of resorcinol capsules versus standard capsules is shown below.

### DETAILED DESCRIPTION OF THE INVENTION

In the process of making a microcapsule product, the melamine-formaldehyde crosslinker used is highly reactive with itself as well as with other polymers possessing amine, amide, carboxyl, enolizable carbonyl, hydroxyl, and thiol moieties. Molecules that possess two or more of these groups are thus capable of participating in crosslinking with melamine-formaldehyde. Unlike melamine-formaldehyde, these molecules are not capable of reacting with themselves. The melamine-formaldehyde crosslinking motif can represented as A-A-A-A whereas with the adjunct crosslinker (B) can be represented by A-A-B-A-B-A-A-A-B, where the adjunct is always surrounded by melamine-formaldehyde, because two adjunct crosslinkers will not link.

By introducing the adjunct crosslinker into the reaction mixture the level of melamine-formaldehyde crosslinker can be reduced while maintaining a high degree of crosslinking and reducing the amount of formaldehyde without compromising wall permeability.

In another embodiment of the invention, an alternate mode of crosslinking is also possible. During the encapsulation process formaldehyde exists in its free form both as a byproduct of the melamine-formaldehyde crosslinking reaction and as contaminate from the melamine-formaldehyde crosslinker raw material. This free formaldehyde can copolymerize with the adjuncts as well. This adjunct-formaldehyde copolymer can either exist by itself, “woven” into the capsule wall (interpenetrating network), or bound in the capsule wall via the melamine-formaldehyde crosslinker.

Additionally, other compounds containing the reactive species mentioned above can act as wall modifiers. Instead of possessing two or more sites of reactivity a wall modifier would only possess one. This wall modifier would react with the melamine-formaldehyde crosslinker and by doing so change the character of the wall either through permeability or hydrophobicity or a combination of both.

A secondary benefit to the adjunct crosslinking is that there is less potential formaldehyde in the capsules. It was mentioned above that the adjunct crosslinking allows for less melamine-formaldehyde crosslinker to be used resulting in less formaldehyde in the system. In addition due to the completeness of the adjunct crosslinking the potential formaldehyde of the melamine-formaldehyde crosslinker is incorporated in the polymer network and is unavailable for release. Thus the adjunct crosslinker has an indirect and direct dual effect on reducing formaldehyde levels.

According to one embodiment of the invention the adjunct crosslinkers can be defined by the following formulas:

(R¹-)mX¹(—R²—Y)n (I)

and

(R¹-)m(-R²—Y)nX¹—R³—X²(—R⁴-Z)o(-R⁵)p (II)

where:
X¹ and X² can be equal to C, N or NH, Phosphate, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate. They can be different in structure.
Y and Z can be selected from amine, amide, carboxyl, enolizable carbonyl, hydroxyl, thiol moieties, and mixture thereof.
R¹ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R² can either not be present or equal to, CH₂, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R³ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R⁴ can be equal to aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.
R⁵ can either not be present or equal to, CH₂, aliphatic moiety, aromatic moiety (benzene, naphthalene, etc.), aliphatic cyclic, partially unsaturated aliphatic cyclic, heteroatom cyclic (i.e. pyridine, imidazol, furan, etc.), carbohydrate, polyalkylene oxide (i.e. PEG and PPG), blocked distributions of 2 or more alkylene oxide monomers.

In structure (I) n is larger than 1 but can be equal or less than the maximum substitution possible on the X group. In addition, m+n is equal or less than the maximum substitution possible on the X group. Finally, the values for m and n are integers or non-integers. Non integer values can arise when distributions of substitution are present. For instance, if X═C, then n can vary between 2 and 4. If X is a benzene ring, then n can vary between 2 and 6.

In structure (II) the values of n and o are at least 1 but can be equal or less than the maximum substitution possible on the X¹ and X² groups, respectively. In addition, m+n is equal or less than the maximum substitution possible on the X¹ group minus 1. Furthermore, o+p is equal or less than the maximum substitution possible on the X² group minus 1. Finally, the values for m, n, o and p are integers or non-integers. Non integer values can arise when distributions of substitution are present. For instance is X¹ and X² are carbon atoms m+n< or =3 and o+p< or =3. If X¹ and X² are nitrogen atoms m+n< or =2 and o+p< or =2.

### Aminoplast-Adjunct Crosslinker Chemistry

Partially methylated, high-imino melamine-formaldehyde crosslinkers like the type used in the capsules undergo the following self-crosslinking reaction:

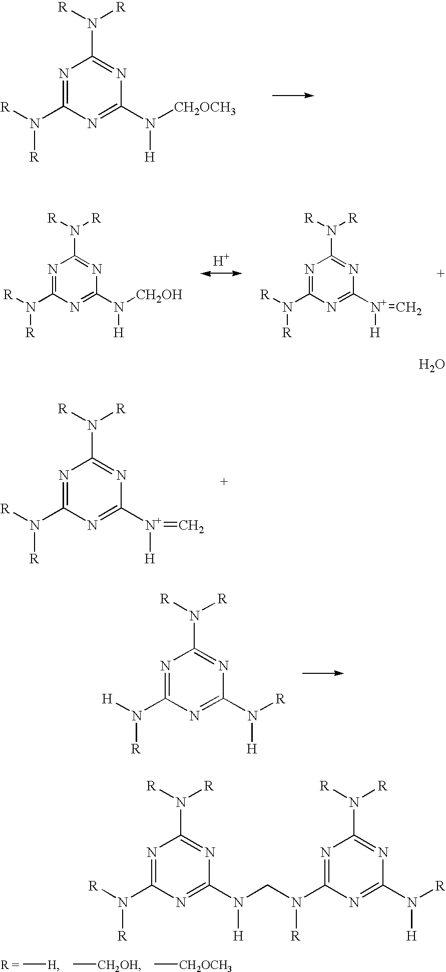

The crosslinker first loses the methoxymethyl group, if present, to form the methylol species. In acidic medium, as found in the capsule-making reaction, the methylol group is in equilibrium with the imino group. It is this imino group which reacts with an amino group on another crosslinker molecule. Thus each crosslinker has two reactive moieties, amino and imino, and there are at least two amino and three imino sites per molecule. Because of the numbers of these groups a three-dimensional crosslinked network is formed.

As mentioned above, melamine-formaldehyde crosslinkers are reactive with themselves as well as with other molecules possessing certain functional groups. For these other molecules to participate in the crosslinking reaction there must be at least two of these groups present per molecule. In the scope of this work the functional groups are of the nucleophilic class. Thus molecules possessing two or more amine, enolizable carbonyl, hydroxyl, and/or thiol moieties can function as adjunct crosslinkers. A general reaction scheme is shown below.

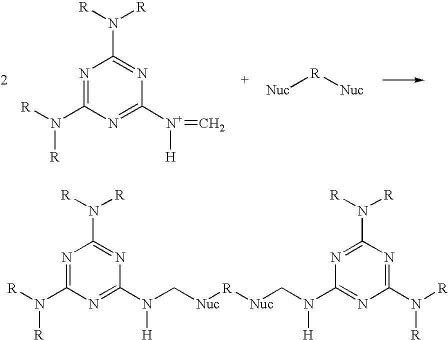

Since there are several crosslinker molecules and adjunct molecules present in a reaction mixture, reactions of this type, in addition to crosslinker-crosslinker reactions, continue on forming a three-dimensional crosslinked network. The degree of adjunct-crosslinker and crosslinker-crosslinker reaction can be controlled by the reactant stoichiometry. When there is more crosslinker than adjunct, crosslinker-crosslinker reactions are favored. When there is more adjunct than crosslinker, adjunct-crosslinker reactions are favored, but only to a certain point. If there is too much adjunct present all the reactive sites on the crosslinker become saturated and no crosslinking is possible, either through the crosslinker or the adjunct. The mole ratio of formaldehyde-based crosslinker to adjunct crosslinker is about 1 to about 500, more preferably about 1 to about 100 and most preferably about 1 to about 50.

As mentioned in the introduction above the adjunct can also copolymerize with the free formaldehyde present.

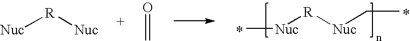

This adjunct formaldehyde copolymer can either coexist in the crosslinked network forming the wall or it can bind to the network in the manner of the adjunct-crosslinker reaction above.

Alternatively the adjunct molecule can act as a wall modifier, capping off the melamine-formaldehyde crosslinker.

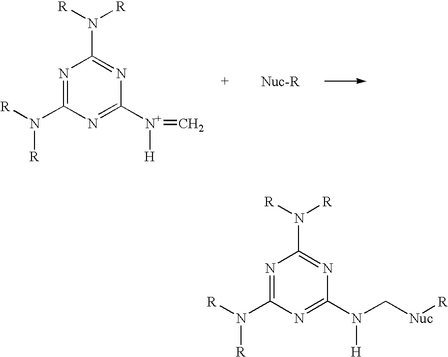

While this may prevent further crosslinking reactions, doing so can change the character of the wall. This depends on the structure and moieties present on the adjunct molecule.

Polyhydroxyl Adjunct Crosslinking with Tris

Tris (hydroxymethyl)amino methane, referred to herein as Tris, can participate in the crosslinking reaction as follows:

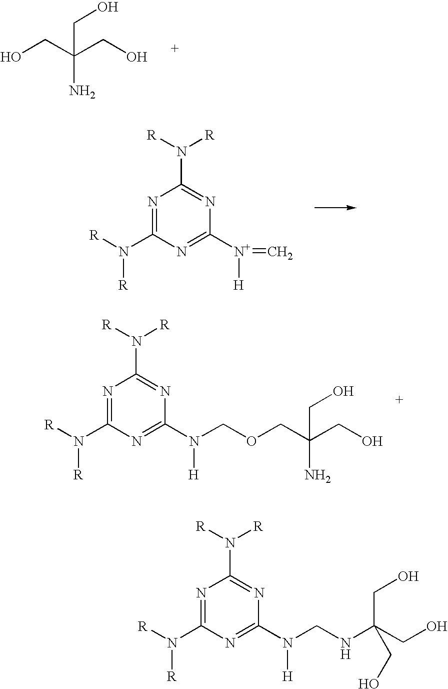

Reaction with the melamine-formaldehyde crosslinker can occur either through the hydroxyl or the amino group of Tris. During the encapsulation process the pH is about 5. At this pH the amino group of Tris would be protonated:

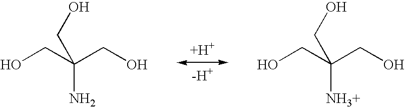

In its protonated form the amino group is not nucleophilic and cannot react with the melamine-formaldehyde crosslinker. Thus in our encapsulation process Tris most likely reacts with the melamine-formaldehyde crosslinker via the hydroxyl groups. Other polyhydroxyls that were tested are shown below:

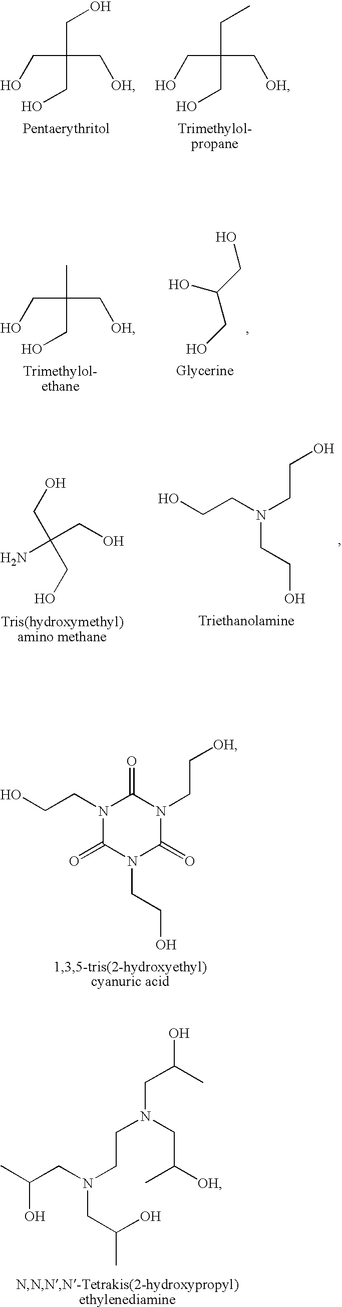

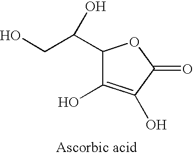

Analogues of hydroxyl groups will also react by the same mechanism and have the same effect on leaking.

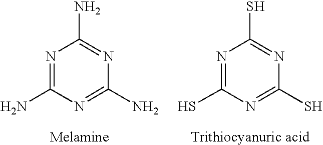

Melamine which essentially possesses the nitrogen analogues of hydroxyl groups (amines) will function as an adjunct crosslinker. The downside is that melamine is not very water soluble so only a small amount can be employed. In addition free melamine is a substance that we are trying to minimize by reducing the crosslinker levels so it doesn't follow that we would add it back in. A molecule containing the sulfur analogues of hydroxyl groups, thiols, is trithiocyanuric acid. It is also the sulfur analogue of melamine and functions as an adjunct crosslinker in the same way.

Beta-Dicarbonyl Wall Reinforcement and Crosslinking with Resorcinol

Resorcinol is an enolizable carbonyl-type adjunct crosslinker whose structure is shown below:

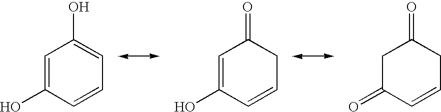

Resorcinol can be considered the enolic form of a diketone and therefore possesses three sites of reactivity per molecule:

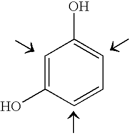

Thus resorcinol can participate in the adjunct crosslinking reaction as follows:

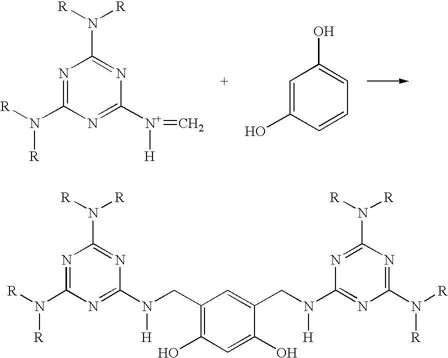

Resorcinol is incapable of reacting with itself so the presence of melamine-formaldehyde crosslinker is crucial.

Other substances that behave like resorcinol and can participate in the crosslinking reaction are shown below.

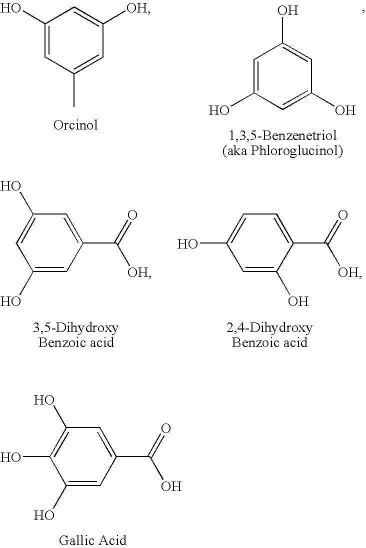

In addition to acting as adjunct crosslinkers 3,5-dihydroxybenzoic acid, 2,4-dihydroxybenzoic acid, and gallic acid can also be used as acidulents in place of acetic acid during the capsule making reaction.

### Scavenger Technology

Several sets of formaldehyde eliminators are disclosed herein, each reacting with formaldehyde by a different mechanism. Formaldehyde eliminators are understood by this invention to include formaldehyde scavengers and reducers and these terms may be used interchangeably.

According to one embodiment, the formaldehyde scavenger can be used from effective trace amounts up to 100 times the stoichiometric amount. The stoichiometric amount is the amount of scavenger required to theoretically bind or react all the formaldehyde added in the form of an aminoplast crosslinker (bound and free formaldehyde). This amount of scavenger can be added either to the slurry or afterward to the final product formulation. For instance, an unscavenged slurry can be added to the formulation, followed by a certain amount of scavenger.

The particular quantity of formaldehyde-based crosslinker that is used to create the capsule slurry contains a percentage of free formaldehyde and bound formaldehyde. The total combined moles of free and bound formaldehyde will determine the amount of moles of scavenger that is needed to react with all the formaldehyde. To drive this reaction to completion we will have to add about a 10× molar excess of scavenger, preferably about a 5× molar excess of scavenger. By moles here is meant moles of scavenging groups. So if the scavenger molecule is multifunctional (i.e. polymeric) less moles of this molecule needs to be added. This is the maximum level of scavenger needed based on the amount of crosslinker used.

The minimum level of scavenger required is that amount that scavenges only the free formaldehyde in the slurry. This level is determined analytically. The minimum amount of moles of scavenger required is equal to the moles of measured formaldehyde (1:1). The reason for determining this minimum level is because the process can affect the level of free formaldehyde in the final slurry. Again, if the scavenger molecule is multifunctional (i.e. polymeric) less moles of this molecule needs to be added.

In a further embodiment, the formaldehyde scavengers disclosed throughout the specification may be added directly to a consumer product. The additional scavenger may be adder from about 0.01 times up to about 100 times the molar amount of all the formaldehyde in the consumer product of The additional scavenger maintains reduced levels of formaldehyde that is subsequently generated during storage by reacting with scavenger, especially in consumer products with a pH less than 3 such as fabric softener.

In the case of multifunctional scavengers such scavenging polymers and solid supports the moles of scavenger in the above specifications is determined by the number of moles scavenging groups added via the polymer or the solid support.

According to the present invention β-dicarbonyl compounds are effective formaldehyde scavengers. The β-dicarbonyl compounds of the present invention have an acidic hydrogen giving rise to a nucleophilic atom that can react with formaldehyde. The β-dicarbonyl compounds contemplated by the present invention are represented by the following structures:

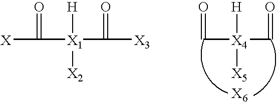

Structure 1a Structure 1b

wherein X, X³ and X⁶ may be selected from the group consisting of H;

(1) a C1-22 straight chain, branched or cyclic hydrocarbon or an aromatic moiety selected from phenyl, phenylene, naphthalene or other polyaromatic hydrocarbons, followed by a polar group or 1-3 halogens. The X, X³ and X⁶ groups may be chemically linked to form cyclic or heterocyclic structures;
(2) a halogen on its own;
(3) a polar group followed by H or a C1-22 straight chain, branched or cyclic hydrocarbon or an aromatic moiety selected from phenyl, phenylene, naphthalene or other polyaromatic hydrocarbon; and
(4) a polar group on its own.

The halogen described in the options above may be selected from F, Cl, Br and I.

The polar group described in the options above may be selected from O, OH, COOH, carbonyl, amide, amine, thiol, quaternary nitrogen ethoxy or propoxy group, or combinations thereof;

and wherein X¹ and X⁴ is either C, N, S, or P;

and wherein X² and X⁵ may be selected from the group consisting of H;

(1) a C1-22 straight chain, branched or cyclic hydrocarbon or aromatic moiety selected from phenyl, phenylene, naphthalene or other polyaromatic hydrocarbons, followed by a polar group or about 1 to about 3 halogens;
(2) a halogen on its own;
(3) a polar group followed by H or a C1-22 straight chain, branched or cyclic hydrocarbon or an aromatic moiety selected from phenyl, phenylene, naphthalene or other polyaromatic hydrocarbon; and
(4) a polar group on its own.

The halogen described in the options above may be selected from F, Cl, Br and I.

The polar group described in the options above may be selected from O, OH, COOH, carbonyl, amide, amine, thiol, quaternary nitrogen ethoxy or propoxy group and combinations thereof.

The β-dicarbonyl scavengers react with formaldehyde by the following reaction scheme:

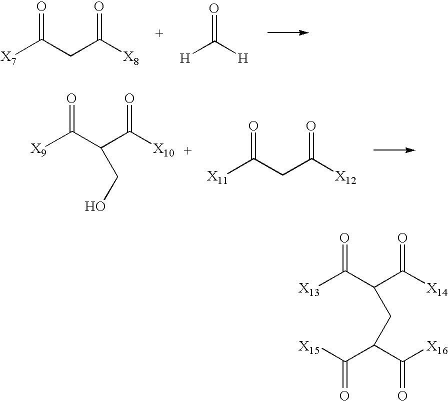

X₇₋₁₆ may be independently selected from the group consisting of H;

(1) a C1-22 straight chain, branched or cyclic hydrocarbon or aromatic moiety selected from phenyl, phenylene, naphthalene and other polyaromatic hydrocarbons, followed by a polar group and about 1 to about 3 halogens;
(2) a halogen on its own;
(3) a polar group followed by H or a C1-22 straight chain, branched or cyclic hydrocarbon or an aromatic moiety selected from phenyl, phenylene, naphthalene or other polyaromatic hydrocarbon; and
(4) a polar group on its own.

The halogen described in the options above may be selected from F, Cl, Br and I.

The polar group described in the options above may be selected from O, OH, COOH, carbonyl, amide, amine, thiol, quaternary nitrogen ethoxy or propoxy group and combinations thereof.

Initially one equivalent of scavenger reacts with one equivalent of formaldehyde resulting in a methylol compound. Another equivalent of scavenger reacts with the methylol carbon forming the stable, disubstituted adduct.

The preferred β-dicarbonyl compounds are acetoacetamide (BKB (Eastman)), ethyl acetoacetate (EAA (Eastman)), N,N-Dimethyleneacetamide (DMAA (Eastman)), acetoacetone, dimethyl-1,3-acetonedicarboxylate, 1,3-acetonedicarboxylic acid, malonic acid, resorcinol, 1,3-cyclohexadione, barbituric acid, 5,5-dimethyl-1,3-cyclohexanedione (dimedone), 2,2-dimethyl-1,3-dioxane-4,6-dione (Meldrum's acid), salicylic acid, methyl acetoacetate (MAA (Eastman)), ethyl-2-methyl acetoacetate, 3-methyl-acetoacetone, dimethyl malonate, diethyl malonate, 1,3-dimethyl barbituric acid, resorcinol, phloroglucinol, orcinol, 2,4-dihydroxy benzoic acid, 3,5-dihydroxy benzoic acid, malonamide and β-dicarbonyl scavenger listed in U.S. Pat. Nos. 5,194,674 and 5,446,195 as well as in Tomasino et al, Textile Chemist and Colorist, vol. 16, No. 12 (1984), which are hereby incorporated by reference.

Mono or Di-Amide scavengers may also be used as effective formaldehyde reducers. The di-amide scavengers are represented by the following structure:

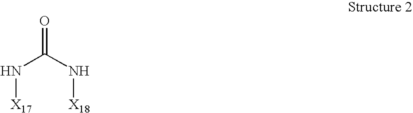

wherein X¹⁷ and X¹⁸ may be independently selected from the group consisting of H;

(1) a C1-22 straight chain, branched or cyclic hydrocarbon or aromatic moiety selected from phenyl, phenylene, naphthalene or other polyaromatic hydrocarbons, followed by a polar group or 1-3 halogens;
(2) a halogen on its own;
(3) a polar group followed by H or a C1-22 hydrocarbon (straight chain, branched or cyclic) or an aromatic moiety (phenyl, phenylene, naphthalene or other polyaromatic hydrocarbon); and
(4) a polar group on its own.

The halogen described in the options above may be selected from F, Cl, Br and I.

According to the present invention, di-amide scavengers react with formaldehyde through the nitrogen and form the following adducts as represented in the below reaction scheme:

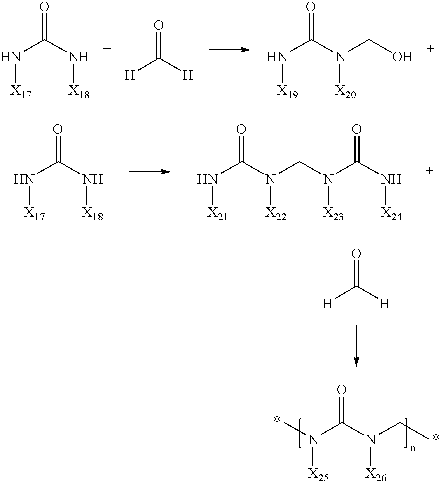

wherein X¹⁷⁻²⁶ may be independently selected from the group consisting of H;
(1) a C1-22 straight chain, branched or cyclic hydrocarbon or aromatic moiety selected from phenyl, phenylene, naphthalene or other polyaromatic hydrocarbons, followed by a polar group or 1-3 halogens;
(2) a halogen on its own;
(3) a polar group followed by H or a C1-22 straight chain, branched or cyclic hydrocarbon or aromatic moiety selected from phenyl, phenylene, naphthalene or other polyaromatic hydrocarbons; and
(4) a polar group.
The halogen described in the options above may be selected from F, Cl, Br and I.

The initial mechanism is similar to the β-dicarbonyl compounds described above. Depending on the functionality of the urea either a disubstituted or polymeric adduct is formed. Examples of the preferred effective mono- and di-amide scavengers are urea, ethylene urea, propylene urea, ε-caprolactam, glycouril, hydantoin, 2-oxazolidinone, 2-pyrrolidinone, uracil, barbituric acid, thymine, uric acid, allantoin, polyamides, 4,5-dihydroxyethylene urea, monomethylol-4-hydroxy-4-methoxy-5,5-dimethyl-propylurea, nylon 2-hydroxyethyl ethylene urea (SR-511; SR-512 (Sartomer)), 2-hydroxyethyl urea (Hydrovance (National Starch)), L-citrulline, biotin, N-methyl urea, N-ethyl urea, N-butyl urea, N-phenyl urea, 4,5-dimethoxy ethylene urea and succinimide.

Another class of compounds that are effective formaldehyde scavengers are amines which form imines by reaction with formaldehyde as represented by the following reaction schemes:

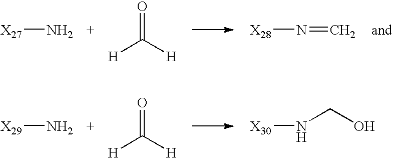

wherein X²⁷⁻³⁰ may be independently selected from the group consisting of H;

(1) a C1-22 straight chain, branched or cyclic hydrocarbon or aromatic moiety selected from phenyl, phenylene, naphthalene or other polyaromatic hydrocarbon followed by a polar group or about 1 to about 3 halogens;
(2) a halogen on its own;
(3) a polar group followed by H or a C1-22 straight chain, branched or cyclic hydrocarbon or an aromatic moiety selected from phenyl, phenylene, naphthalene and other polyaromatic hydrocarbon; and
(4) a polar group on its own.

The halogen described in the options above may be selected from F, Cl, Br and I.

Depending upon the amine, similar but different products may be obtained.

Preferred amines contemplated by this invention include, but are not limited to, poly(vinyl amine) (Lupamin (BASF)), arginine, lysine, asparagines, proline, tryptophan, 2-amino-2-methyl-1-propanol (AMP); proteins such as casein, gelatin, collagen, whey protein, soy protein, and albumin; melamine, benzoguanamine, 4-aminobenzoic acid (PABA), 3-aminobenzoic acid, 2-aminobenzoic acid (anthranilic acid), 2-aminophenol, 3-aminophenol, 4-aminophenol, creatine, 4-aminosalicylic acid, 5-aminosalicylic acid, methyl anthranilate, methoxylamine HCl, anthranilamide, 4-aminobenzamide, p-toluidine, p-anisidine, sulfanilic acid, sulfanilamide, methyl-4-aminobenzoate, ethyl-4-aminobenzoate (benzocain), beta-diethylaminoethyl-4-aminobenzoate (procain), 4-aminobenzamide, 3,5-diaminobenzoic acid and 2,4-diaminophenol. Other amines as disclosed in copending U.S. Letters for patent application Ser. No. 11/123,898 and U.S. Pat. No. 6,261,483, and those mentioned in Tomasino et al, Textile Chemist and Colorist, vol. 16, No. 12 (1984), are also contemplated by the present invention and hereby incorporated by reference. Hydrazines such as 2,4-dinitrophenzylhydrazine can also react with formaldehyde by the first method to give hydrazones. The reaction is pH-dependent and reversible. Other preferred amines can be selected from a non-limiting list of 1,2-phenylenediamine, 1,3-phenylenediamine, and 1,4-phenylenediamine. In addition, aromatic amines, triamines, and aliphatic polyamine may also be used. Examples of these amines may include, but are not limited to, aniline, hexamethylenediamine, bis-hexamethylenetriamine, triethylaminetriamine, poly(propyleneoxide)triamine, and poly(propyleneglycol)diamines.

Another class of formaldehyde reducers provided by the present invention is acetal forming compounds such as those represented by the following structure:

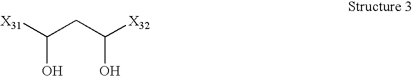

wherein X³¹ and X³² may be independently selected from the group consisting of H;

(1) a C1-22 straight chain, branched or cyclic hydrocarbon or aromatic moiety selected from phenyl, phenylene, naphthalene or other polyaromatic hydrocarbons, followed by a polar group or 1-3 halogens;
(2) a halogen on its own;
(3) a polar group followed by H or a C1-22 straight chain, branched or cyclic hydrocarbon or aromatic moiety selected from phenyl, phenylene, naphthalene or other polyaromatic hydrocarbons; and
(4) a polar group on its own.
The halogen described in the options above may be selected from F, Cl, Br, or I.

Preferred acetal forming compounds include, but are not limited to, diethylene glycol, saccharides such as D-sorbitol and sucrose, tannins/tannic acid, and polysaccharides such as starches, guar, xanthan, pectin, chemically-modified cellulose, chitosan, absorbic acid, dextrose and mixtures thereof. Also suitable are aliphatic alcohols listed in Tomasino et al, Textile Chemist and Colorist, vol. 16, No. 12 (1984), which is hereby incorporated by reference. Furthermore, polymers with alcohol functional groups such as polyvinylalcohol may be selected.

The acetal forming complex reacts with formaldehyde according to the following general reaction scheme:

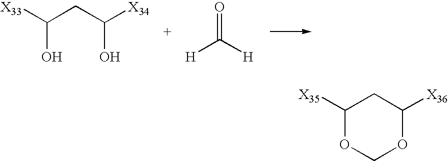

wherein X³³⁻³⁶ may be independently selected from the group consisting of H;

(1) a C1-22 straight chain, branched or cyclic hydrocarbon or aromatic moiety selected from phenyl, phenylene, naphthalene or other polyaromatic hydrocarbons, followed by a polar group or 1-3 halogens;
(2) a halogen on its own;
(3) a polar group followed by H or a C1-22 straight chain, branched or cyclic hydrocarbon or an aromatic moiety selected from phenyl, phenylene, naphthalene and other polyaromatic hydrocarbon; and
(4) a polar group on its own.

The halogen described in the options above may be selected from F, Cl, Br and I.

Similar to the amines described above the reaction is pH-dependent and reversible.

Sulfur containing compounds are also capable of reacting with and scavenging formaldehyde. There are two modes of reaction. The first reaction is with a bisulfite:

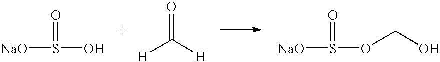

In this case the formaldehyde reacts with the sulfur-bound oxygen forming a stable addition compound. Preferred sulfur containing compounds are, but not limited to, 1,3,5-triazine-2,4,6-trithiol (TAICROS TMT; TMT 15 (DeGussa)) and glutathione.

The other mode of reaction is similar to the acetal mechanism above, with the sulfur groups taking the place of the oxygens.

A unique case is with the amino acid cysteine which has vicinal sulfur and nitrogen groups and illustrated in the following reaction scheme:

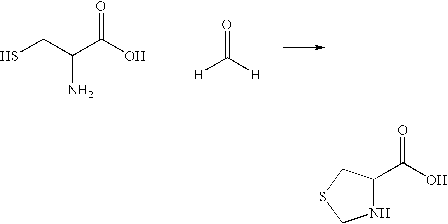

Cysteine forms a stable complex with formaldehyde. Proteins containing cysteine can also participate in this reaction.

### Scavengers Immobilized on Solid Supports

According to one embodiment of the invention, scavenger moieties can be attached to the surfaces of solid supports. Solid supports are defined as substances that are insoluble in the capsule slurry, the product base containing the capsule slurry and the product base in use. The solid support scavengers can be added to the capsule slurry or a commercial product containing capsules to reduce formaldehyde levels. The formaldehyde becomes permanently bound to the solid support and results in an adduct that is inert and benign.

Suitable solid supports can be polymeric or inorganic in nature. Examples of polymeric supports are polyolefins such as polyethylene, polystyrene, polyvinylacetate, polysaccharides such as dextran, poly esters, polyamides, polyurethanes, polyacrylates and polyureas. The polymers can be straightchained, branched or crosslinked. The surfaces of the supports can be further treated to allow the attachment the scavenger moieties. An example of such treatment is the oxidation by plasma.

Examples of inorganic supports are clays, alumina, silica, zeolite and titanium dioxide. The supports can range in size from sub-micron to millimeter dimension. Bound scavenger moieties that are effective are aromatic amines, thiol, thiourea, urea and beta-dicarbonyls. The scavengers can exist bound directly to the support or bound to a linker molecule that is then bound directly to the support. A commercial example of a thiol functional resin is Amberlite/Duolite GT-73 (Rohm & Haas). Commercial examples of thiourea functional resins are Lewatit MonoPlus TP-214 (Lanxess) and Ionac SR-3 (Lanxess). Beta-dicarbonyl, aromatic amine and thiol functional resins are sold by Sigma-Aldrich.

### Polymeric Scavengers

In another embodiment of the invention, polymeric scavengers can be added to the capsule slurry or the product base to scavenge formaldehyde. Polymeric scavengers are defined as macromolecular species that contain scavenging moieties. Additionally, polymeric scavengers are soluble in the capsule slurry, the product base containing the capsule slurry and the product base in use. Scavenger moieties can be attached to the polymeric backbones as either endgroups or pendant groups. Alternatively, the polymer backbone can contain scavenging moieties. The advantage of a polymeric scavenger is that the scavenger and the scavenger-formaldehyde adduct are macromolecular and therefore typically more inert and benign.

Suitable polymer backbones which may be modified with scavenging endgroups are based on vinyl, acrylic, olefin, saccharide, alkylene-oxide, amine, urea, urethane, carbonate, ester, and amide/peptide chemistries. The polymer molecular weights can range in size from 100 to 10,000,000 Daltons (more preferably from 500 to 1,000,000 Daltons). The polymers can be straight-chained, branched, crosslinked and networked in structure. The scavenging moieties can be attached to either all or some of the chain-ends. End-group scavenger moieties that are effective, can be selected from, but not limited to, are amides, ureas, thiols, sulfites, aromatic amines and beta-dicarbonyls. Examples of endgroup polymeric scavengers are poly(1,4-butanediol)-bis-(4-aminobenzoate) and poly(ethyleneglycol) diacetoacetate. Scavenging moieties can be present in the polymer from 0.1 to 100 weight percent.

There are two types of scavenging pendant groups. One type is where the existing pendant has inherent scavenging ability. This would be when the pendant is terminated with an N, O, or S moiety. Suitable polymer backbones are those based on vinyl amine, vinyl alcohol, vinyl mercaptan and allylamine. Other pendant scavenger moieties that are effective are, but not limited to, aromatic amines, ureas, thiols and beta-dicarbonyls. The polymer molecular weights can range in size from 100 to 10,000,000 Daltons and more preferably from 500 to 1,000,000 Daltons. The polymers can be straight-chained, branched, or crosslinked or networked in structure. Examples of pendant polymeric scavengers are poly(vinyl amine) (Lupamin (BASF)) and poly(vinyl alcohol).

Another type of pendant scavenger group is where the existing pendant functional group is further functionalized by reaction to yield pendants with scavenging ability. In this way a polymer with pendant groups devoid of scavenging activity can be converted to an effective scavenger. Suitable polymer backbones are those based on acrylic acid, methacrylic acid, maleic anhydride, maleic acid, itaconic acid, acrylamide, vinyl amine, vinyl alcohol, vinyl mercaptan, saccharides, peptides, and allylamine. The polymer molecular weights can range in size from 100 to 10,000,00 Daltons (more preferably from 500 to 1,000,000 Daltons). The polymers can be straight-chained, branched, or crosslinked or networked in structure.

The scavenging moieties can be attached to all or some of the pendant groups. The scavengers can exist bound directly to the pendant group or bound to a linker molecule that this then bound directly to the pendant group. Suitable scavenger moieties are selected from, but not limited to, amines, amides/ureas, thiols, and beta-dicarbonyls. Those skilled in the art can determine the specific reaction pathway for attaching these scavenging moieties to the pendant groups.

In addition to the above mentioned formaldehyde reducers, formaldehyde may also be removed (i.e. elimination or absorption) to achieve partial and complete formaldehyde removal. As stated before, the formaldehyde scavenger can be used from trace amounts up to 100 times the stoichiometric amount. The stoichiometric amount is the amount of scavenger required to theoretically bind or react all the formaldehyde added in the form of an aminoplast crosslinker (bound and free formaldehyde).

The material can be added either during the capsule making process, after the capsules are formed or both. Process conditions do affect the efficiency of the scavenging reaction. This pH is to be selected from about 1 to about 9, more preferably from about 2 to about 8, most preferably from about 2 to about 6.

The optimum conditions, such as pH and temperature, are highly dependent on the scavenging chemistry. Nevertheless, often more suitable pH conditions are above and below 7. In addition, higher temperature conditions may often be favorable.

The stability of capsules may be affected when scavengers are used. One way to minimize this effect is to utilize a combination of scavengers such as, but not limited to, the combination of urea and ethylene urea to maintain lower formaldehyde levels and stability. For such scavenger combinations, which may be 2 or more, each of the individual scavengers can be present at 0.1-99.9% of the total amount of scavenger added (the combination as a whole). For example, a suitable combination would be urea and ethylene urea in the ratio 1:3 to 3:1. Such combinations include the option to have a scavenger or scavenger combination used in the capsule slurry as well as a different scavenger or scavenger combination added to the final consumer product.

Another embodiment of this invention is to remove formaldehyde or formaldehyde-scavenger adducts from the capsule slurry using a solid support such as commercially available active carbon. This is surprising as formaldehyde is very water soluble. The active carbon can be washed and reused. The activated carbon can be selected from any commercial sources prepared from a wide range of processes using coal, wood and coconut. Granular activated carbon is preferred over powder samples for easy handling. Some non-limiting examples are TIGG 5D 1240, TIGG 5DR 0840, TIGG 5D 2050, TIGG 5WCS-G, and TIGG 5DAW 1240 from TIGG Corporation (Bridgeville, Pa.); GC 8×30, GC 8×30AW, GC 8×30S, GC 12×40SAW from General Carbon Corp., (Paterson, N.J. 07501); and CAL® 12×40, FILTRASORB® 100&200, and FILTERSOB 300&400® from Calgon Carbon (Pittsburgh, Pa.). A more extensive list may be found in the technique brochures published by manufactures. The activated carbon can be added to the formaldehyde solution at the same time the formaldehyde-adduct is formed. It can also be added at a later stage.

In a variant of the above embodiment, formaldehyde may be removed by ammonization and the formed adducts may be subsequently adsorbed with activated carbon. According to this embodiment, formaldehyde reacts with ammonium in alkaline medium to form hemethyleneteramine which may then be adsorbed by activated carbon.

Another embodiment of invention is to remove formaldehyde from the capsule slurry by direct oxidation:

(1) to produce formic acid. Formaldehyde is removed after oxidized to formic acid with hydrogen peroxide in an alkaline base to form formic acid/salt complex.

(2) to carbon dioxide. Here, formaldehyde is oxidized to carbon dioxide by exhaustive chemical oxidation and thus removed from capsule slurry. This can be achieved by the oxidation of formaldehyde by H₂O₂ in acidic medium. Optionally, bleach activators and/or bleach catalysts (including oxidizing enzymes) may be used to speed up the oxidation. Detailed options for this application are listed below.

The oxidation reaction of formaldehyde can be facilitated by using a transition metal ion such as iron (II) or iron (III) as catalyst. Redox-active transition metal ions such as Cu(I) and Mn (II) may also be used. Enzymes such peroxidase may also be utilized.

It is also possible to remove formaldehyde by chemical oxidation using manganese oxide (MnO₂). Formaldehyde may be oxidized by MnO₂ in acid medium and thus removed from capsule slurry. Other inorganic or organic oxidizer may include, but not limited to, ruthenium oxide (RuO₂), vanadium oxide (V₂O₅), sodium percarbonate, permanganate, sodium perborate. The amount of oxidizer should be enough to react stoichiometrically with the amount of formaldehyde originally present in the unscavenged slurry. That unscavenged slurry formaldehyde level depends on the formaldehyde level added to the slurry via the aminoplast crosslinker.

In order to optimize the oxidation various bleach sources may be used. These may optionally be accelerated and activated using bleach activators and catalysts (synthetic and enzymatic). The options are listed below.

### Bleach Sources

Hydrogen peroxide (H₂O₂), hypochlorite, chlorine, peracids, oxygen, ozone, and chlorine dioxygen.

### H₂O₂Sources

Hydrogen peroxide sources are listed in Kirk Othmer's Sons), Vol 4, pp. 271-300 “Bleaching Agents (Survey)”. Some of the sources of hydrogen peroxide are sodium perborate, sodium percarbonate, sodium carbonate peroxyhydrate, sodium pyrophosphate peroxyhydrate, urea peroxyhydrate, or sodium peroxide can be used herein. Another useful source of available oxygen is persulfate bleach (e.g., OXONE, manufactured by DuPont).

### Bleach Activators

These materials can activate the release of peroxide. Examples of these are: TAED (tetraacetylethylenediamine). Other activators are listed in U.S. Pat. No. 4,915,854, issued Apr. 10, 1990 to Moa et al, and U.S. Pat. No. 4,412,934. Also, nonanolyloxybenzene sulfonate (NOBS) or acyl lactam activators may be used, and mixtures thereof with TAED can also be used. Conventional bleach activators are listed in U.S. Pat. No. 4,634,551. Another class of bleach activators are amido-derived bleach activators which are described in U.S. Pat. No. 4,634,551. Also, bleach activators comprising the benzoxazin-type activators disclosed by Hodge et al in U.S. Pat. No. 4,966,723, can be used. Furthermore, bleach activators of the class of acyl lactam activators such as octanoyl caprolactam, 3,5,5-trimethylhexanoyl caprolactam, nonanoyl caprolactam, decanoyl caprolactam, valerolactam, undecenoyl valerolactam, nonanoyl valerolactam, 3,5,5-trimethylhexanoyl valerolactam and mixtures thereof can be used.

Finally, quaternary substituted bleach activators may be used such as those disclosed in U.S. patent applications 298,903, 298,650, 298,906 and 298,904, incorporated herein by reference.

### Bleach Catalysts

Bleach catalysts can be use to further catalyze the bleaching/oxidizing reaction. Examples of such catalysts are: transition metal cation salts and complexes with organic reagents; metal salts being manganese, cobalt, copper, iron, titanium, ruthenium, tungston, and molybdenum. Cobalt complex catalysts as disclosed in EP application 408,131. Also catalysts of lower metals can be used (disclosed in U.S. Pat. No. 4,430,243).

Manganese-based complexes disclosed in U.S. Pat. Nos. 5,246,621 and 5,244,594, EP Application 549,272, and U.S. Pat. No. 5,194,416.

Complexes with other ligands such as 1,5,9-trimethyl-1,5,9-triazacyclododecane, 2-methyl-1,4,7-triazacyclononane, 2-methyl-1,4,7-triazacyclononane, and mixtures thereof.

Metal salt complex with a non-carboxylate polyhydroxy compound having at least three consecutive C—OH groups, such as those disclosed in U.S. Pat. No. 5,114,606. For example, complexes of manganese (II), (III), and/or (IV) with sorbitol, iditol, dulsitol, mannitol, xylithol, arabitol, adonitol, meso-erythritol, meso-inositol, lactose, and mixtures thereof.

Bleach catalysts of the type described in U.S. Pat. No. 5,114,611. Examples are bleach catalysts comprising Co, Cu, Mn, Fe, -bispyridylmethane and -bispyridylamine complexes such as Co(2,2′-bispyridylamine)Cl₂, Di(isothiocyanato)bispyridylamine-cobalt (II), trisdipyridylamine-cobalt(II) perchlorate, Co(2,2-bispyridylamine)₂O₂ClO₄, Bis-(2,2′-bispyridylamine) copper(II) perchlorate, tris(di-2-pyridylamine) iron(II) perchlorate, and mixtures thereof.

Mn gluconate, Mn(CF₃SO₃)₂, Co(NH₃)₅Cl, and the binuclear Mn complexed with tetra-N-dentate and bi-N-dentate ligands, including N₄Mn^{III}(u-O)₂Mn^{IV} N₄)⁺ and [Bipy₂Mn^{III}(u-O)₂ Mn^{IV} bipy₂]-(ClO₄)₃.

Metallo porphyrin catalysts such as those disclosed in EP Application Nos. 384,503, and 306,089.

Absorbed catalysts onto mineral supports such as disclosed in U.S. Pat. Nos. 4,601,845 and 4,711,748.

Bleach catalysts that are disclosed in U.S. Pat. Nos. 4,728,455, 4,711,748, 4,626,373, 4,119,557, 4,430,243, 4,728,455 and DE Patent No. 2,054,019. Another group of bleach catalysts that may be used are the polyoxymetallates.

### Oxidizing Enzymes

Oxidizing enzymes such as horseradish peroxidase, haloperoxidases, amine oxidase, amino acid oxidase, cholesterol oxidase, uric acid oxidase, xanthine oxidase, glucose oxidase, galactose oxidase and alcohol oxidase may also be used to oxidize formaldehyde.

The concentration of oxidizer needed can be calculated by the concentration of formaldehyde used. The molar ratio of peroxide to formaldehyde can vary from 1 to 20, preferably 1 to 10. The amount of catalyst can be used at level where a reasonable rate is achieved. A preferably ratio will be one tenth to 1% of that of the peroxide.

It is appreciated by those skilled in the art that the formaldehyde eliminators described above may be used alone or in combination with the formaldehyde absorbers described above. The ratio formaldehyde absorber (active carbon) to slurry is determined by the level of formaldehyde present. That means that prior to formaldehyde absorption, one skilled in the art should evaluate the binding capacity of the formaldehyde absorber and make sure that the capacity for formaldehyde absorption is in excess of the amount of formaldehyde in the unscavenged capsule slurry.

Process pH and temperature conditions for employing oxidizing agents depends on the type of bleach source. More moderate conditions are possible when using bleach activators and catalysts (synthetic or enzymes).

In another embodiment of the invention, the formaldehyde scavengers disclosed herein can be used in a process to increase the stability of a microcapsule product by curing the microcapsules at higher temperatures. The retention capabilities of the microcapsule product are improved when the crosslinked network of polymers containing active materials are cured at temperatures above 90° C. In a more preferred embodiment the retention capabilities of microcapsule product are improved when the cure temperature is above 110° C. In a most preferred embodiment the retention capabilities of the microcapsule product are improved when the cure temperature is above 120° C. In a further embodiment the crosslinked network of polymers containing active materials may be cured for periods of time longer than 1 hour and more preferably longer than two hours.

The term high stability refers to the ability of a microcapsule product to retain active materials in bases that have a tendency to promote leaching of the active material out of the microcapsule product into the base. For example, there exists a relationship between higher concentration of surfactants in the base of consumer products and an increased leaching effect of the encapsulated active materials out of the microcapsules and into the base. Bases that are primarily non-aqueous in nature, e.g., those that are based on alcohols, or volatile silicones can also leach active materials from capsules over time. Volatile silicones such as but not limited to Cyclomethicone and are exemplified by SF1256 Cyclopentasiloxane, SF1257 Cyclopentasiloxane are trademarks of General Electric Company. Volatile silicones are in a number of personal care products, such as antiperspirants, deodorants, hair sprays, cleansing creams, skin creams, lotions and stick products, bath oils, suntan and shaving product, make-up and nail polishes. In these product types, the base solvent itself solubilizes the active material.

### Capsule Technology

Encapsulation of active materials such as fragrances is known in the art, see for example U.S. Pat. Nos. 2,800,457, 3,870,542, 3,516,941, 3,415,758, 3,041,288, 5,112,688, 6,329,057, and 6,261,483. Another discussion of fragrance encapsulation is found in the Kirk-Othmer Encyclopedia.

Preferred encapsulating polymers include those formed from melamine-formaldehyde or urea-formaldehyde condensates, as well as similar types of aminoplasts. Additionally, microcapsules made via the simple or complex coacervation of gelatin are also preferred for use with the coating. Microcapsules having shell walls comprised of polyurethane, polyamide, polyolefin, polysaccaharide, protein, silicone, lipid, modified cellulose, gums, polyacrylate, polystyrene, and polyesters or combinations of these materials are also functional.

A representative process used for aminoplast encapsulation is disclosed in U.S. Pat. No. 3,516,941 though it is recognized that many variations with regard to materials and process steps are possible. A representative process used for gelatin encapsulation is disclosed in U.S. Pat. No. 2,800,457 though it is recognized that many variations with regard to materials and process steps are possible. Both of these processes are discussed in the context of fragrance encapsulation for use in consumer products in U.S. Pat. Nos. 4,145,184 and 5,112,688 respectively.

Well known materials such as solvents, surfactants, emulsifiers, and the like can be used in addition to the polymers described throughout the invention to encapsulate the active materials such as fragrance without departing from the scope of the present invention. It is understood that the term encapsulated is meant to mean that the active material is substantially covered in its entirety. Encapsulation can provide pore vacancies or interstitial openings depending on the encapsulation techniques employed. More preferably the entire active material portion of the present invention is encapsulated.

Fragrance capsules known in the art consists of a core of various ratios of fragrance and solvent materials, a wall or shell comprising a three-dimensional cross-linked network of an aminoplast resin, more specifically a substituted or un-substituted acrylic acid polymer or co-polymer cross-linked with a urea-formaldehyde pre-condensate or a melamine-formaldehyde pre-condensate.

Microcapsule formation using mechanisms similar to the foregoing mechanism, using (i) melamine-formaldehyde or urea-formaldehyde pre-condensates and (ii) polymers containing substituted vinyl monomeric units having proton-donating functional group moieties (e.g. sulfonic acid groups or carboxylic acid anhydride groups) bonded thereto is disclosed in U.S. Pat. No. 4,406,816 (2-acrylamido-2-methyl-propane sulfonic acid groups), UK published Patent Application GB 2,062,570 A (styrene sulfonic acid groups) and UK published Patent Application GB 2,006,709 A (carboxylic acid anhydride groups).

The cross-linkable acrylic acid polymer or co-polymer microcapsule shell wall precursor has a plurality of carboxylic acid moieties, to with:

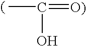

and is preferably one or a blend of the following:
(i) an acrylic acid polymer;
(ii) a methacrylic acid polymer;
(iii) an acrylic acid-methacrylic acid co-polymer;
(iv) an acrylamide-acrylic acid co-polymer;
(v) a methacrylamide-acrylic acid co-polymer;
(vi) an acrylamide-methacrylic acid co-polymer;
(vii) a methacrylamide-methacrylic acid co-polymer;
(viii) a C₁-C₄ alkyl acrylate-acrylic acid co-polymer;
(ix) a C₁-C₄ alkyl acrylate-methacrylic acid co-polymer;
(x) a C₁-C₄ alkyl methacrylate-acrylic acid co-polymer;
(xi) a C₁-C₄ alkyl methacrylate-methacrylic acid co-polymer;
(xii) a C₁-C₄ alkyl acrylate-acrylic acid-acrylamide co-polymer;
(xiii) a C₁-C₄ alkyl acrylate-methacrylic acid-acrylamide co-polymer;
(xiv) a C₁-C₄ alkyl methacrylate-acrylic acid-acrylamide co-polymer;
(xv) a C₁-C₄ alkyl methacrylate-methacrylic acid-acrylamide co-polymer;
(xvi) a C₁-C₄ alkyl acrylate-acrylic acid-methacrylamide co-polymer;
(xvii) a C₁-C₄ alkyl acrylate-methacrylic acid-methacrylamide co-polymer;
(xviii) a C₁-C₄ alkyl methacrylate-acrylic acid-methacrylamide co-polymer; and
(xix) a C₁-C₄ alkyl methacrylate-methacrylic acid-methacrylamide co-polymer;
   and more preferably, an acrylic acid-acrylamide copolymer.

When substituted or un-substituted acrylic acid co-polymers are employed in the practice of our invention, in the case of using a co-polymer having two different monomeric units, e.g. acrylamide monomeric units and acrylic acid monomeric units, the mole ratio of the first monomeric unit to the second monomeric unit is in the range of from about 1:9 to about 9:1, preferably from about 3:7 to about 7:3. In the case of using a co-polymer having three different monomeric units, e.g. ethyl methacrylate, acrylic acid and acrylamide, the mole ratio of the first monomeric unit to the second monomeric unit to the third monomeric unit is in the range of 1:1:8 to about 8:8:1, preferably from about 3:3:7 to about 7:7:3.

The molecular weight range of the substituted or un-substituted acrylic acid polymers or co-polymers useful in the practice of our invention is from about 5,000 to about 1,000,000, preferably from about 10,000 to about 100,000. The substituted or un-substituted acrylic acid polymers or co-polymers useful in the practice of our invention may be branched, linear, star-shaped, dendritic-shaped or may be a block polymer or copolymer, or blends of any of the aforementioned polymers or copolymers.

Such substituted or un-substituted acrylic acid polymers or co-polymers may be prepared according to any processes known to those skilled in the art, for example, U.S. Pat. No. 6,545,084.

The urea-formaldehyde and melamine-formaldehyde pre-condensate microcapsule shell wall precursors are prepared by means of reacting urea or melamine with formaldehyde where the mole ratio of melamine or urea to formaldehyde is in the range of from about 10:1 to about 1:6, preferably from about 1:2 to about 1:5. For purposes of practicing our invention, the resulting material has a molecular weight in the range of from 156 to 3000. The resulting material may be used ‘as-is’ as a cross-linking agent for the aforementioned substituted or un-substituted acrylic acid polymer or copolymer or it may be further reacted with a C₁-C₆ alkanol, e.g. methanol, ethanol, 2-propanol, 3-propanol, 1-butanol, 1-pentanol or 1-hexanol, thereby forming a partial ether where the mole ratio of melamine or urea:formalhyde:alkanol is in the range of 1:(0.1-6):(0.1-6). The resulting ether moiety-containing product may by used ‘as-is’ as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or co-polymers. Methods for formation of such melamine-formaldehyde and urea-formaldehyde pre-condensates are set forth in U.S. Pat. No. 3,516,846, U.S. Pat. No. 6,261,483, and Lee et al. J. Microencapsulation, 2002, Vol. 19, No. 5, pp 559-569, “Microencapsulation of fragrant oil via in situ polymerization: effects of pH and melamine-formaldehyde molar ratio”. Examples of urea-formaldehyde pre-condensates useful in the practice of our invention are URAC 180 and URAC 186, trademarks of Cytec Technology Corp. of Wilmington, Del. 19801, U.S.A. Examples of melamine-formaldehyde pre-condensates useful in the practice of our invention are CYMEL U-60, CYMEL U-64 and CYMEL U-65, trademarks of Cytec Technology Corp. of Wilmington, Del. 19801, U.S.A. In the practice of our invention it is preferable to use as the precondensate for cross-linking the substituted or un-substituted acrylic acid polymer or co-polymer. The melamine-formaldehyde pre-condensate having the structure:

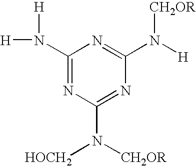

wherein each of the R groups are the same or different and each represents hydrogen or C₁-C₆ lower alkyl, e.g. methyl, ethyl, 1-propyl, 2-propyl, 1-butyl, 2-butyl, 2-methyl-1-propyl, 1-pentyl, 1-hexyl and/or 3-methyl-1-pentyl.

In practicing our invention, the range of mole ratios of urea-formaldehyde precondensate or melamine-formaldehyde pre-condensate: substituted or un-substituted acrylic acid polymer or co-polymer is in the range of from about 9:1 to about 1:9, preferably from about 5:1 to about 1:5 and most preferably from about 2:1 to about 1:2.

In one embodiment of the invention, capsules with polymer(s) comprising primary and/or secondary amine reactive groups or mixtures thereof and crosslinkers are provided. The amine polymers can possess primary and/or secondary amine functionalities and can be of either natural or synthetic origin. Amine containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable amine polymers are the Lupamin series of polyvinyl formamides (available from BASF). The molecular weights of these materials can range from 10,000 to 1,000,000.

The polymers containing primary and/or secondary amines can be used with any of the following comonomers in any combination:
1. Vinyl and acrylic monomers with:
   a. alkyl, aryl and silyl substituents;
   b. OH, COOH, SH, aldehyde, trimonium, sulfonate, NH₂, NHR substituents;
   c. vinyl pyridine, vinyl pyridine-N-oxide, vinyl pyrrolidon
2. Cationic monomers such as dialkyl dimethylammonium chloride, vinyl imidazolinium halides, methylated vinyl pyridine, cationic acrylamides and guanidine-based monomers
3. N-vinyl formamide
   and any mixtures thereof. The ratio amine monomer/total monomer ranges from 0.01-0.99 and more preferred from 0.1-0.9.

The following represents a general formula for the amine-containing polymer material:

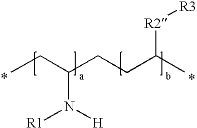

wherein R is a saturated or unsaturated alkane, dialkylsiloxy, dialkyloxy, aryl, alkylated aryl, and that may further contain a cyano, OH, COOH, NH₂, NHR, sulfonate, sulphate, —NH₂, quaternized amines, thiols, aldehyde, alkoxy, pyrrolidone, pyridine, imidazol, imidazolinium halide, guanidine, phosphate, monosaccharide, oligo or polysaccharide.

R¹ is H, CH₃, (C═O)H, alkylene, alkylene with unsaturated C—C bonds, CH₂—CROH, (C═O)—NH—R, (C═O)—(CH₂)n-OH, (C═O)—R, (CH₂)n-E, —(CH₂—CH(C═O))n-XR, —(CH₂)n-COOH, —(CH₂)n-NH₂, —CH₂)n-(C═O)NH₂, E is an electrophilic group; wherein a and b are integers or average numbers (real numbers) from about 100-25,000.

R² can be nonexistent or the functional group selected from the group consisting of —COO—, —(C═O)—, —O—, —S—, —NH—(C═O)—, —NR1-, dialkylsiloxy, dialkyloxy, phenylene, naphthalene, alkyleneoxy. R³ can be the same or selected from the same group as R¹.

Additional copolymers with amine monomers are provided having the structure:

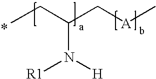

R¹ is H, CH₃, (C═O)H, alkylene, alkylene with unsaturated C—C bonds, CH₂—CROH, (C═O)—NH—R, (C═O)— (CH₂)n-OH, (C═O)—R, (CH₂)n-E, —(CH₂—CH(C═O)) n-XR, —(CH₂)n-COOH, —(CH₂)n-NH₂, —CH₂) n-(C═O)NH₂, E is an electrophilic group; wherein a and b are integers or average numbers (real numbers) from about 100-25,000.

The comonomer, represented by A, can contain an amine monomer and a cyclic monomer wherein A can be selected from the group consisting of aminals, hydrolyzed or non-hydrolyzed maleic anhydride, vinyl pyrrolidine, vinyl pyridine, vinyl pyridine-N-oxide, methylated vinyl pyridine, vinyl naphthalene, vinyl naphthalene-sulfonate and mixtures thereof.

When A is an aminal the following general structure can represent the aminal:

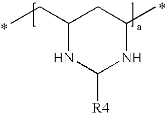

wherein R⁴ is selected from the group consisting of H, CH₃, (C═O)H, alkylene, alkylene with unsaturated C—C bonds, CH₂—CROH, (C═O)—NH—R, (C═O)—(CH₂)n-OH, (C═O)—R, (CH₂)n-E, —(CH₂—CH(C═O))n-XR, —(CH₂)n-COOH, —(CH₂)n-NH2, —CH₂)n-(C═O)NH₂, E is an electrophilic group; wherein R is a saturated or unsaturated alkane, dialkylsiloxy, dialkyloxy, aryl, alkylated aryl, and that may further contain a cyano, OH, COOH, NH₂, NHR, sulfonate, sulphate, —NH₂, quaternized amines, thiols, aldehyde, alkoxy, pyrrolidone, pyridine, imidazol, imidazolinium halide, guanidine, phosphate, monosaccharide, oligo or polysaccharide.

In addition instead of amine-containing polymers it is possible to utilize amine-generating polymers that can generate primary and secondary amines during the capsule formation process.

Once the fragrance material is encapsulated a cationically charged water-soluble polymer may optionally be applied to the fragrance encapsulated polymer. This water-soluble polymer can also be an amphoteric polymer with a ratio of cationic and anionic functionalities resulting in a net total charge of zero and positive, i.e., cationic. Those skilled in the art would appreciate that the charge of these polymers can be adjusted by changing the pH, depending on the product in which this technology is to be used. Any suitable method for coating the cationically charged materials onto the encapsulated fragrance materials can be used. The nature of suitable cationically charged polymers for assisted capsule delivery to interfaces depends on the compatibility with the capsule wall chemistry since there has to be some association to the capsule wall. This association can be through physical interactions, such as hydrogen bonding, ionic interactions, hydrophobic interactions, electron transfer interactions or, alternatively, the polymer coating could be chemically (covalently) grafted to the capsule or particle surface. Chemical modification of the capsule or particle surface is another way to optimize anchoring of the polymer coating to capsule or particle surface. Furthermore, the capsule and the polymer need to want to go to the desired interface and, therefore, need to be compatible with the chemistry (polarity, for instance) of that interface. Therefore, depending on which capsule chemistry and interface (e.g., cotton, polyester, hair, skin, wool) is used the cationic polymer can be selected from one or more polymers with an overall zero (amphoteric: mixture of cationic and anionic functional groups) or net positive charge, based on the following polymer backbones: polysaccharides, polypeptides, polycarbonates, polyesters, polyolefinic (vinyl, acrylic, acrylamide, poly diene), polyester, polyether, polyurethane, polyoxazoline, polyamine, silicone, polyphosphazine, olyaromatic, poly heterocyclic, or polyionene, with molecular weight (MW) ranging from about 1,000 to about 1000,000,000, preferably from about 5,000 to about 10,000,000. As used herein molecular weight is provided as weight average molecular weight. Optionally, these cationic polymers can be used in combination with nonionic and anionic polymers and surfactants, possibly through coacervate formation.

A more detailed list of cationic polymers that can be used to coat the encapsulated fragrance is provided below: Polysaccharides include but are not limited to guar, alginates, starch, xanthan, chitosan, cellulose, dextrans, arabic gum, carrageenan, hyaluronates. These polysaccharides can be employed with:
(a) cationic modification and alkoxy-cationic modifications, such as cationic hydroxyethyl, cationic hydroxy propyl. For example, cationic reagents of choice are 3-chloro-2-hydroxypropyl trimethylammonium chloride or its epoxy version. Another example is graft-copolymers of polyDADMAC on cellulose like in Celquat L-200 (Polyquaternium-4), Polyquaternium-10 and Polyquaternium-24, commercially available from National Starch, Bridgewater, N.J.;
(b) aldehyde, carboxyl, succinate, acetate, alkyl, amide, sulfonate, ethoxy, propoxy, butoxy, and combinations of these functionalities. Any combination of Amylose and Mylopectin and overall molecular weight of the polysaccharide; and
(c) any hydrophobic modification (compared to the polarity of the polysaccharide backbone).

The above modifications described in (a), (b) and (c) can be in any ratio and the degree of functionalization up to complete substitution of all functionalizable groups, and as long as the theoretical net charge of the polymer is zero (mixture of cationic and anionic functional groups) or preferably positive. Furthermore, up to 5 different types of functional groups may be attached to the polysaccharides. Also, polymer graft chains may be differently modified than the backbone. The counterions can be any halide ion or organic counter ion. U.S. Pat. No. 6,297,203 and U.S. Pat. No. 6,200,554.

Another source of cationic polymers contain protonatable amine groups so that the overall net charge is zero (amphoteric: mixture of cationic and anionic functional groups) or positive. The pH during use will determine the overall net charge of the polymer. Examples are silk protein, zein, gelatin, keratin, collagen and any polypeptide, such as polylysine.

Further cationic polymers include poly vinyl polymers, with up to 5 different types of monomers, having the monomer generic formula —C(R²)(R¹)—CR²R³—. Any co-monomer from the types listed in this specification may also be used. The overall polymer will have a net theoretical positive charge or equal to zero (mixture of cationic and anionic functional groups). Where R¹ is any alkanes from C₁-C₂₅ or H; the number of double bonds ranges from 0-5. Furthermore, R¹ can be an alkoxylated fatty alcohol with any alkoxy carbon-length, number of alkoxy groups and C₁-C₂₅ alkyl chain length. R¹ can also be a liquid crystalline moiety that can render the polymer thermotropic liquid crystalline properties, or the alkanes selected can result in side-chain melting. In the above formula R² is H or CH₃; and

R³ is —C₁, —NH₂ (i.e., poly vinyl amine or its copolymers with N-vinyl formamide. These are sold under the name Lupamin 9095 by BASF Corporation), —NHR¹, —NR¹R², —NR¹R²R⁶ (where R⁶═R¹, R² or —CH₂—COOH or its salt), —NH—C(O)—H, —C(O)—NH₂ (amide), —C(O)—N(R²)(R²′)(R²″), —OH, styrene sulfonate, pyridine, pyridine-N-oxide, quaternized pyridine, imidazolinium halide, imidazolium halide, imidazol, piperidine, pyrrolidone, alkyl-substituted pyrrolidone, caprolactam or pyridine, phenyl-R⁴ or naphthalene-R⁵ where R⁴ and R⁵ are R¹, R², R³, sulfonic acid or its alkali salt —COOH, —COO— alkali salt, ethoxy sulphate or any other organic counter ion. Any mixture or these R³ groups may be used. Further suitable cationic polymers containing hydroxy alkyl vinyl amine units, as disclosed in U.S. Pat. No. 6,057,404.

Another class of materials is polyacrylates, with up to 5 different types of monomers, having the monomer generic formula: —CH(R1)-C(R2)(CO—R3-R4)—. Any co-monomer from the types listed in this specification may also be used. The overall polymer will have a net theoretical positive charge or equal to zero (mixture of cationic and anionic functional groups). In the above formula R¹ is any alkane from C₁-C₂₅ or H with number of double bonds from 0-5, aromatic moieties, polysiloxane, or mixtures thereof. Furthermore, R¹ can be an alkoxylated fatty alcohol with any alkoxy carbon-length, number of alkoxy groups and C1-C25 alkyl chain length. R¹ can also be a liquid crystalline moiety that can render the polymer thermotropic liquid crystalline properties, or the alkanes selected can result in side-chain melting. R² is H or CH₃; R³ is alkyl alcohol C₁-C₂₅ or an alkylene oxide with any number of double bonds, or R³ may be absent such that the C═O bond is (via the C-atom) directly connected to R⁴. R⁴ can be: —NH₂, NHR¹, —NR¹R², —NR¹R²R⁶ (where R⁶═R¹, R², or CH₂ COOH or its salt), —NH—C(O)—, sulfo betaine, betaine, polyethylene oxide, poly(ethyleneoxide/propylene oxide/butylene oxide) grafts with any end group, H, OH, styrene sulfonate, pyridine, quaternized pyridine, alkyl-substituted pyrrolidone or pyridine, pyridine-N-oxide, imidazolinium halide, imidazolium halide, imidazol, piperidine, —OR¹, —OH, —COOH alkali salt, sulfonate, ethoxy sulphate, pyrrolidone, caprolactam, phenyl-R⁴ or naphthalene-R⁵ where R⁴ and R⁵ are R¹, R², R³, sulfonic acid or its alkali salt or organic counter ion. Any mixture or these R3 groups may be used. Also, glyoxylated cationic polyacrylamides can be used. Typical polymers of choice are those containing the cationic monomer dimethylaminoethyl methacrylate (DMAEMA) or methacrylamidopropyl trimethyl ammonium chloride (MAPTAC). DMAEMA can be found in Gafquat and Gaffix VC-713 polymers from ISP. MAPTAC can be found in BASF's Luviquat PQ11 PN and ISP's Gafquat HS100.

Another group of polymers that can be used are those that contain cationic groups in the main chain or backbone. Included in this group are:
(1) polyalkylene imines such as polyethylene imine, commercially available as Lupasol from BASF. Any molecular weight and any degree of crosslinking of this polymer can be used in the present invention;
(2) ionenes having the general formula set forth as —[N(+)R¹R²-A¹-N(R⁵)—X—N(R⁶)-A²-N(+)R³R⁴-A]n-2Z- as disclosed in U.S. Pat. No. 4,395,541 and U.S. Pat. No. 4,597,962;
(3) adipic acid/dimethyl amino hydroxypropyl diethylene triamine copolymers, such as Cartaretin F-4 and F-23, commercially available from Sandoz;
(4) polymers of the general formula —[N(CH₃)²—(CH₂)ₓ—NH—(CO)—NH—(CH₂)y-N(CH₃)²)—(CH₂)z-O— (CH₂)p]n-, with x, y, z, p=1-12, and n according to the molecular weight requirements. Examples are Polyquaternium 2 (Mirapol A-15), Polyquaternium-17 (Mirapol AD-1), and Polyquaternium-18 (Mirapol AZ-1).

Other polymers include cationic polysiloxanes and cationic polysiloxanes with carbon-based grafts with a net theoretical positive charge or equal to zero (mixture of cationic and anionic functional groups). This includes cationic end-group functionalized silicones (i.e. Polyquaternium-80). Silicones with general structure: —[—Si(R¹)(R²)—O-]x-[Si(R³)(R²)—O-]_{y}— where R¹ is any alkane from C₁-C₂₅ or H with number of double bonds from 0-5, aromatic moieties, polysiloxane grafts, or mixtures thereof. R¹ can also be a liquid crystalline moiety that can render the polymer thermotropic liquid crystalline properties, or the alkanes selected can result in side-chain melting. R² can be H or CH₃ and

R³ can be —R¹-R⁴, where R4 can be —NH₂, —NHR¹, —NR¹R², —NR¹R²R⁶ (where R⁶═R¹, R², or —CH₂—COOH or its salt), —NH—C(O)—, —COOH, —COO— alkali salt, any C₁-C₂₅ alcohol, —C(O)—NH₂ (amide), —C(O)—N(R²)(R²′) (R²″), sulfo betaine, betaine, polyethylene oxide, poly(ethyleneoxide/propylene oxide/butylene oxide) grafts with any end group, H, —OH, styrene sulfonate, pyridine, quaternized pyridine, alkyl-substituted pyrrolidone or pyridine, pyridine-N-oxide, imidazolinium halide, imidazolium halide, imidazol, piperidine, pyrrolidone, caprolactam, —COOH, —COO— alkali salt, sulfonate, ethoxy sulphate phenyl-R⁵ or naphthalene-R⁶ where R⁵ and R⁶ are R¹, R², R³, sulfonic acid or its alkali salt or organic counter ion. R³ can also be —(CH₂)x-O—CH₂—CH(OH)—CH₂—N(CH₃)²—CH₂—COOH and its salts. Any mixture of these R³ groups can be selected. X and y can be varied as long as the theoretical net charge of the polymer is zero (amphoteric) or positive. In addition, polysiloxanes containing up to 5 different types of monomeric units may be used. Examples of suitable polysiloxanes are found in U.S. Pat. No. 4,395,541 U.S. Pat. No. 4,597,962 and U.S. Pat. No. 6,200,554. Another group of polymers that can be used to improve capsule/particle deposition are phospholipids that are modified with cationic polysiloxanes. Examples of these polymers are found in U.S. Pat. No. 5,849,313, WO Patent Application 9518096A1 and European Patent EP0737183B1.

Furthermore, copolymers of silicones and polysaccharides and proteins can be used (commercially available as CRODASONE brand products).

Another class of polymers include polyethylene oxide-co-propyleneoxide-co-butylene oxide polymers of any ethylene oxide/propylene oxide/butylene oxide ratio with cationic groups resulting in a net theoretical positive charge or equal to zero (amphoteric). The general structure is:

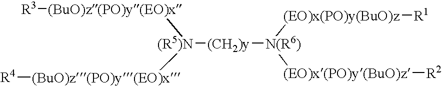

where R¹,R²,R³,R⁴ is —NH₂, —N(R)³—X+, wherein R being H or any alkyl group. R⁵,R⁶ is —CH₃ or H. Counter ions can be any halide ion or organic counter ion. X, Y, may be any integer, any distribution with an average and a standard deviation and all 12 can be different. Examples of such polymers are the commercially available TETRONIC brand polymers.

Suitable polyheterocyclic (the different molecules appearing in the backbone) polymers include the piperazine-alkylene main chain copolymers disclosed in Ind. Eng. Chem. Fundam., (1986), 25, pp. 120-125, by Isamu Kashiki and Akira Suzuki.

Also suitable for use in the present invention are copolymers containing monomers with cationic charge in the primary polymer chain. Up to 5 different types of monomers may be used. Any co-monomer from the types listed in this specification may also be used. Examples of such polymers are poly diallyl dimethyl ammonium halides (PolyDADMAC) copolymers of DADMAC with vinyl pyrrolidone, acrylamides, imidazoles, imidazolinium halides, etc. These polymers are disclosed in Henkel EP0327927A2 and PCT Patent Application 01/62376A1. Also suitable are Polyquaternium-6 (Merquat 100), Polyquaternium-7 (Merquats S, 550, and 2200), Polyquaternium-22 (Merquats 280 and 295) and Polyquaternium-39 (Merquat Plus 3330), available from Ondeo Nalco.

Polymers containing non-nitrogen cationic monomers of the general type —CH₂—C(R¹)(R²—R³—R⁴)— can be used with:

R¹ being a —H or C₁-C₂₀ hydrocarbon. R² is a disubstituted benzene ring or an ester, ether, or amide linkage. R³ is a C₁-C₂₀ hydrocarbon, preferably C₁-C₁₀, more preferably C₁-C₄. R⁴ can be a trialkyl phosphonium, dialkyl sulfonium, or a benzopyrilium group, each with a halide counter ion. Alkyl groups for R4 are C₁-C₂₀ hydrocarbon, most preferably methyl and t-butyl. These monomers can be copolymerized with up to 5 different types of monomers. Any co-monomer from the types listed in this specification may also be used.

Substantivity of these polymers may be further improved through formulation with cationic, amphoteric and nonionic surfactants and emulsifiers, or by coacervate formation between surfactants and polymers or between different polymers. Combinations of polymeric systems (including those mentioned previously) may be used for this purpose as well as those disclosed in EP1995/000400185.

Furthermore, polymerization of the monomers listed above into a block, graft or star (with various arms) polymers can often increase the substantivity toward various surfaces. The monomers in the various blocks, graft and arms can be selected from the various polymer classes listed in this specification and the sources below:
Encyclopedia of Polymers and Thickeners for Cosmetics, Robert Lochhead and William From, in Cosmetics & Toiletries, Vol. 108, May 1993, pp. 95-138;
Modified Starches: Properties & Uses, O. B. Wurzburg, CRC Press, 1986. Specifically, Chapters 3, 8, and 10;
U.S. Pat. Nos. 6,190,678 and 6,200,554; and
PCT Patent Application WO 01/62376A1 assigned to Henkel.

Polymers, or mixtures of the following polymers:
(a) Polymers comprising reaction products between polyamines and (chloromethyl) oxirane or (bromomethyl) oxirane. Polyamines being 2(R1)N—[—R2-N(R1)-]n—R2-N(R1)2, 2HN—R1-NH2, 2HN—R2-N(R1)2 and 1H-Imidazole. Also, the polyamine can be melamine. R1 in the polyamine being H or methyl. R2 being alkylene groups of C1-C20 or phenylen groups. Examples of such polymers are known under the CAS numbers 67953-56-4 and 68797-57-9. The ratio of (chloromethyl) oxirane to polyamine in the cationic polymer ranges from 0.05-0.95.
(b) Polymers comprising reaction products of alkanedioic acids, polyamines and (chloromethyl) oxirane or (bromomethyl) oxirane. Alkane groups in alkanedioic acids C0-C20.

Polyamine structures are as mentioned in (a). Additional reagents for the polymer are dimethyl amine, aziridine and polyalkylene oxide (of any molecular weight but, at least, di-hydroxy terminated; alkylene group being C1-20, preferably C2-4). The polyalkylene oxide polymers that can also be used are the Tetronics series. Examples of polymers mentioned here are known under the CAS numbers 68583-79-9 (additional reagent being dimethyl amine), 96387-48-3 (additional reagent being urea), and 167678-45-7 (additional reagents being polyethylene oxide and aziridine). These reagents can be used in any ratio.
(c) Polyamido Amine and Polyaminoamide-epichlorohydrin resins, as described by David Devore and Stephen Fisher in Tappi Journal, vol. 76, No. 8, pp. 121-128 (1993). Also referenced herein is “Polyamide-polyamine-epichlorohydrin resins” by W. W. Moyer and R. A. Stagg in Wet-Strength in Paper and Paperboard, Tappi Monograph Series No. 29, Tappi Press (1965), Ch. 3, 33-37.

The preferred cationically charged materials comprise reaction products of polyamines and (chloromethyl) oxirane. In particular, reaction products of 1H-imidazole and (chloromethyl) oxirane, known under CAS number 68797-57-9. Also preferred are polymers comprising reaction products of 1,6-hexanediamine,N-(6-aminohexyl) and (chloromethyl) oxirane, known under CAS number 67953-56-4. The preferred weight ratio of the imidazole polymer and the hexanediamine, amino hexyl polymer is from about 5:95 to about 95:5 weight percent and preferably from about 25:75 to about 75:25.

The level of outer cationic polymer is from about 1% to about 3000%, preferably from about 5% to about 1000% and most preferably from about 10% to about 500% of the fragrance containing compositions, based on a ratio with the fragrance on a dry basis.

The weight ratio of the encapsulating polymer to fragrance is from about 1:25 to about 1:1. Preferred products have had the weight ratio of the encapsulating polymer to fragrance varying from about 1:10 to about 4:96.

For example, if a capsule blend has 20 weight % fragrance and 20 weight % polymer, the polymer ratio would be (20/20) multiplied by 100(%)=100%.

The present invention, the encapsulated fragrance is well suited for wash-off products. Wash-off products are understood to be those products that are applied for a given period of time and then are removed. These products are common in areas such as laundry products, and include detergents, fabric conditioners, and the like; as well as personal care products which include shampoos, hair rinses, body washes, soaps and the like.

As described herein, the present invention is well suited for use in a variety of well-known consumer products such as laundry detergent and fabric softeners, liquid dish detergents, automatic dish detergents, as well as hair shampoos and conditioners. These products employ surfactant and emulsifying systems that are well known. For example, fabric softener systems are described in U.S. Pat. Nos. 6,335,315, 5,674,832, 5,759,990, 5,877,145, 5,574,179, 5,562,849, 5,545,350, 5,545,340, 5,411,671, 5,403,499, 5,288,417, 4,767,547 and 4,424,134. Liquid dish detergents are described in U.S. Pat. Nos. 6,069,122 and 5,990,065; automatic dish detergent products are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562. Liquid laundry detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Shampoo and conditioners that can employ the present invention include U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090, 4,705,681.

### Encapsulated Active Materials

The active material suitable for use in the present invention can be a wide variety of materials in which one would want to deliver in a controlled-release manner onto the surfaces being treated with the present compositions or into the environment surrounding the surfaces. Non-limiting examples of active materials include perfumes, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and the like.

In a preferred embodiment, the active material is a fragrance, in which case the microcapsules containing fragrance provide a controlled-release scent onto the surface being treated or into the environment surrounding the surface. In this case, the fragrance can be comprised of a number of fragrance raw materials known in the art, such as essential oils, botanical extracts, synthetic fragrance materials, and the like.

The level of fragrance in the cationic polymer coated encapsulated fragrance varies from about 5 to about 95 weight percent, preferably from about 40 to about 95 and most preferably from about 50 to about 90 weight percent on a dry basis. In addition to the fragrance other agents can be used in conjunction with the fragrance and are understood to be included.

As noted above, the fragrance may also be combined with a variety of solvents which serve to increase the compatibility of the various materials, increase the overall hydrophobicity of the blend, influence the vapor pressure of the materials, or serve to structure the blend. Solvents performing these functions are well known in the art and include mineral oils, triglyceride oils, silicone oils, fats, waxes, fatty alcohols, diisodecyl adipate, and diethyl phthalate among others.

As described herein, the present invention is well suited for use in a variety of well-known consumer products such as laundry detergent and fabric softeners, liquid dish detergents, automatic dish detergents, as well as hair shampoos and conditioners. These products employ surfactant and emulsifying systems that are well known. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, and 4,767,547, 4,424,134. Liquid dish detergents are described in U.S. Pat. Nos. 6,069,122 and 5,990,065; automatic dish detergent products are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562. Liquid laundry detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681. All of the above mentioned U.S. patents.

All U.S. patents and patent applications cited herein are incorporated by reference as if set forth herein in their entirety.

These and additional modifications and improvements of the present invention may also be apparent to those with ordinary skill in the art. The particular combinations of elements described and illustrated herein are intended only to represent only a certain embodiment of the present invention and are not intended to serve as limitations of alternative articles within the spirit and scope of the invention. All materials are reported in weight percent unless noted otherwise. As used herein all percentages are understood to be weight percent.

The ASTM (American Standards and Testing Methods) test method was used in the following Examples to determine the level of formaldehyde present in the capsule slurries. This standard is issued under the fixed designation D 5910-96.

The following examples are provided as specific embodiments of the present invention. Other modifications of this invention will be readily apparent to those skilled in the art. Such modifications are understood to be within the scope of this invention. As used herein all percentages are weight percent unless otherwise noted, ppm is understood to stand for parts per million and g is understood to be grams. IFF as used in the examples is understood to mean International Flavors & Fragrances Inc., New York, N.Y., USA.

### EXAMPLE I

### Preparation of Microcapsules with Tris Adjunct Crosslinker

A reactor is charged with 44 g of Superfloc A2870M (Kemira) and 288 g of water. 9 g Cymel 385 (Cytec) and 1 g tris (Fisher) are added. This mixture is stirred until a clear solution is obtained. Acetic acid is added until pH 5 is reached. This mixture is then stirred for 2 hours at 23° C. until a Brookfield viscosity of 75 cP is reached. At this point 210 g of the fragrance core consisting of 105 g of fragrance and 105 g of Neobee M-5 oil is added and the mixture high-sheared until a mean droplet size of 8 μm is reached. The temperature is raised to 90° C. for 2 hours to cure the microcapsules. After cooling a white slurry is obtained.

### EXAMPLE II

### Preparation of Microcapsule Product with Resorcinol Adjunct Crosslinkers

A reactor is charged with 44 g of Superfloc A2870M (Kemira) and 293 g of water. 9 g Cymel 385 (Cytec) and 3.7 g resorcinol (Aldrich) are added. This mixture is stirred until a clear solution is obtained. Acetic acid is added until pH 5 is reached. This mixture is then stirred for 1 hour at 23° C. until a Brookfield viscosity of 75 cP is reached. At this point 210 g of the fragrance core consisting of 105 g of fragrance and 105 g of Neobee M-5 oil is added and the mixture high-sheared until a mean droplet size of 8 μm is reached. The temperature is raised to 90° C. for 2 hours to cure the microcapsules. After cooling a white slurry is obtained.

We claim:

1. A microcapsule product comprising an active material; the active material encapsulated by a polymeric material to provide a polymer encapsulated material wherein said polymeric material comprises an adjunct crosslinker represented by the following formula:
(R¹-)mX¹(—R²—Y)n (I)
wherein
X¹ is selected from the group consisting of C, N or NH, Phosphate, aliphatic moiety, aromatic moiety, aliphatic cyclic, partially unsaturated aliphatic cyclic, heteroatom cyclic, carbohydrate;
Y is selected from the group consisting of an amine, amide, carboxyl, enolizable carbonyl, hydroxyl, thiol moieties, and mixture thereof;
R¹ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R² is equal to zero or selected from the group consisting of CH2, aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers; and
with the proviso in structure (I) n is larger than 1 but can be equal or less than the maximum substitution possible on the X group; m+n is equal to or less than the maximum substitution possible on the X group and wherein the values of m and n are integers or non-integers.

2. The microcapsule product comprising an active material;
said active material encapsulated by a polymeric material to provide a polymer encapsulated material wherein said polymeric material comprises an adjunct crosslinker represented by the following formula:
(R¹-)m(-R²—Y)nX¹—R³—X²(—R-Z)o(-R⁵)p (II)
wherein
X¹ and X² is independently selected from the group consisting of C, N or NH, Phosphate, aliphatic moiety, aromatic moiety, aliphatic cyclic, partially unsaturated aliphatic cyclic, heteroatom cyclic, carbohydrate;
Y and Z is independently selected from the group consisting of an amine, amide, carboxyl, enolizable carbonyl, hydroxyl, thiol moieties, and mixture thereof;
R¹ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R² is equal to zero or selected from the group consisting of CH₂, an aliphatic moiety, an aromatic moiety, aliphatic cyclic, partially unsaturated aliphatic cyclic, heteroatom cyclic, carbohydrate, polyalkylene oxide, blocked distributions of 2 or more alkylene oxide monomers;
R³ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R⁴ is selected from the group consisting of an aliphatic moiety, an aromatic moiety, aliphatic cyclic, partially unsaturated aliphatic cyclic, heteroatom cyclic, carbohydrate, polyalkylene oxide, blocked distributions of 2 or more alkylene oxide monomers;
R⁵ is equal to or selected from the group consisting of CH₂, aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
with the proviso in structure (II) the value of n and o are at least 1 but can be equal to or less than the maximum substitution possible on the X¹ and X² groups, respectively, m+n is equal or less than the maximum substitution possible on the X¹ group minus 1, o+p is equal or less than the maximum substitution possible on the X² group minus 1, and wherein the values for m, n, o and p are integers or non-integers.

3. The microcapsule product of claim 1 wherein the adjunct crosslinker is selected from the groups consisting of 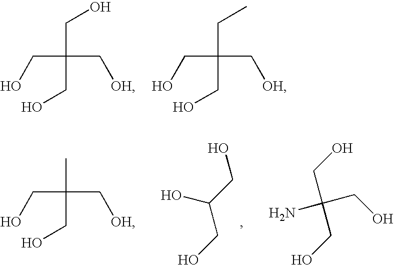
and mixtures thereof.

4. The microcapsule product of claim 3 wherein the adjunct
crosslinker is

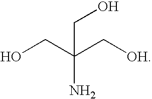

5. The microcapsule product of claim 1 wherein the adjunct crosslinker is selected from the group consisting of 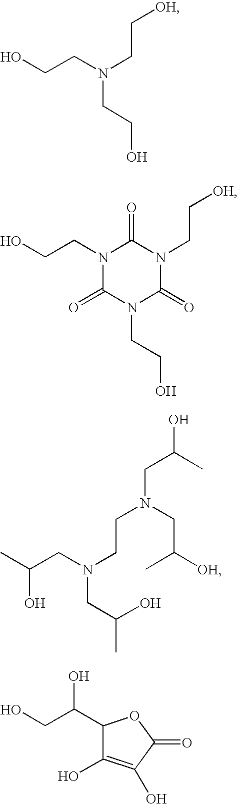
and mixtures thereof.

6. The microcapsule product of claim 1 wherein the adjunct crosslinker is selected from the group consisting of 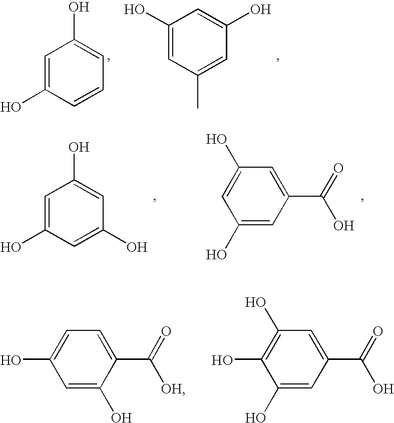
and mixtures thereof.

7. The microcapsule product of claim 7 wherein the adjunct crosslinker is resorcinal.

8. The microcapsule product of claim 1 wherein the polymeric material comprises from a vinyl polymer; an acrylate polymer, melamine-formaldehyde; urea formaldehyde and mixtures thereof.

9. The microcapsule product of claim 8 wherein the mole ratio of melamine-formaldehyde to adjunct crosslinker is in the range of from about 500 to about 0.1.

10. The microcapsule product of claim 8 wherein the mole ratio of melamine-formaldehyde to adjunct crosslinker is in the range of from about 100 to about 0.5.

11. The microcapsule product of claim 8 wherein the mole ratio of melamine-formaldehyde to adjunct crosslinker is in the range of from about 50 to about 1.

12. The microcapsule product of claim 1 wherein the active material is selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

13. The microcapsule product of claim 12 wherein said active material is a fragrance.

14. The microcapsule product of claim 12 wherein said composition further comprises a malodour counteractant composition.

15. The microcapsule product of claim 14 wherein said malodour counteractant composition is selected from the group consisting of uncomplexed cyclodextrin; odor blockers; reactive aldehydes; flavanoids; zeolites; activated carbon; and mixtures thereof.

16. The microcapsule product of claim 1 wherein the polymer encapsulated material is further coated with a cationically charged polymer.

17. The microcapsule product of claim 1 which is incorporated into a product selected from the group consisting of a personal care, fabric care and cleaning products.

18. The composition of claim 17 wherein the personal care product is selected from the group consisting of hair shampoos, hair rinses, hair colors and dyes, bar soaps, and body washes.

19. The microcapsule product of claim 2 wherein the adjunct crosslinker is N,N,N′,N′-tetrakis(2-hydroxypropyl)ethylenediamine.

20. The microcapsule product of claim 2 wherein the mole ratio of melamine-formaldehyde to adjunct crosslinker is in the range of from about 500 to about 0.1.

21. The microcapsule product of claim 2 wherein the mole ratio of melamine-formaldehyde to adjunct crosslinker is in the range of from about 100 to about 0.5.

22. The microcapsule product of claim 2 wherein the mole ratio of melamine-formaldehyde to adjunct crosslinker is in the range of from about 50 to about 1.

23. The microcapsule product of claim 2 wherein the active material is selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

24. The microcapsule product of claim 22 wherein said active material is a fragrance.

25. The microcapsule product of claim 22 wherein said composition further comprises a malodour counteractant composition.

26. The microcapsule product of claim 24 wherein said malodour counteractant composition is selected from the group consisting of uncomplexed cyclodextrin; odor blockers; reactive aldehydes; flavanoids; zeolites; activated carbon; and mixtures thereof.

27. The microcapsule product of claim 2 wherein the polymer encapsulated material is further coated with a cationically charged polymer.

28. The microcapsule product of claim 2 which is incorporated into a product selected from the group consisting of a personal care, fabric care and cleaning products.

29. The composition of claim 27 wherein the personal care product is selected from the group consisting of hair shampoos, hair rinses, hair colors and dyes, bar soaps, and body washes.

30. A process for preparing a microcapsule product, comprising encapsulating an active material with a polymeric material comprises providing an aqueous slurry of a plurality of microcapsules having a polymeric wall and a core comprising an active material, wherein the polymeric material comprises an adjunct crosslinker of claim 1.

31. The process of claim 30 wherein the encapsulating polymer is selected from a vinyl polymer; an acrylate polymer, melamine-formaldehyde; urea formaldehyde and mixtures thereof.

32. The process of claim 29 wherein the weight percentage (%) of adjunct crosslinker present in the wall polymer is from about 0.1 to about 25%.

33. The process of claim 29 wherein the weight percentage (%) of adjunct crosslinker present in the wall polymer is from about 0.1 to about 10%.

34. The process of claim 29 wherein the microcapsule product is further coated by a cationic polymer.

35. The process of claim 33 wherein the cationic polymer is selected from polysaccharides, cationically modified starch and cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides, imidazolium halides and mixtures.

36. The process of claim 34 wherein the cationic polymer is selected from a cationically modified starch, cationically modified guar and mixtures thereof.

37. The process of claim 29 wherein the active material is selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

38. A method of imparting an olfactory effective amount of a fragrance into a consumer product comprising the steps of incorporating at least about 0.25 weight percent (%) of the microcapsule product of claim 1 into a consumer product.

39. The method of claim 37 wherein the consumer product is selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, automatic dishwashing compositions, foodstuffs, beverages and mixtures thereof.

40. A consumer product selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, and automatic dishwashing compositions, foodstuffs, beverages and mixtures thereof comprising the microcapsule product according to the process of claim 29.

41. A process for preparing a microcapsule product, comprising providing an aqueous slurry of a plurality of microcapsules having a polymeric wall and a core comprising an active material, wherein the polymeric material comprises an adjunct crosslinker of claim 2.

42. The process of claim 40 wherein the adjunct is N,N,N′,N′-tetrakis(2-hydroxypropyl)ethylenediamine.

43. The process of claim 40 herein the encapsulating polymer is selected from a vinyl polymer; an acrylate polymer, melamine-formaldehyde; urea formaldehyde and mixtures thereof.

44. The process of claim 40 herein the weight percentage (%) of adjunct crosslinker present in the wall polymer is from about 0.1 to about 25%.

45. The process of claim 40 herein the weight percentage (%) of adjunct crosslinker present in the wall polymer is from about 0.1 to about 10%.

46. The process of claim 40 herein the microcapsule product is further coated by a cationic polymer.

47. The process of claim 45 wherein the cationic polymer is selected from polysaccharides, cationically modified starch and cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides, imidazolium halides and mixtures.

48. The process of claim 46 wherein the cationic polymer is selected from a cationically modified starch, cationically modified guar and mixtures thereof.

49. The process of claim 40 wherein the active material is selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

50. A method of imparting an olfactory effective amount of a fragrance into a consumer product comprising the steps of incorporating at least about 0.25 weight % of the microcapsule product of claim 2 into a consumer product.

51. The method of claim 49 wherein the consumer product is selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, automatic dishwashing compositions, foodstuffs, beverages and mixtures thereof.

52. A consumer product selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, and automatic dishwashing compositions, foodstuffs, beverages and mixtures thereof comprising the microcapsule product according to the process of claim 50.

53. A process for preparing a microcapsule product with reduced levels of free formaldehyde, which comprises:
a) providing a plurality of microcapsules comprising a polymeric wall, an adjunct crosslinker and a core comprising an active material, wherein the microcapsules comprises formaldehyde;
b) providing a stoichiometric excess of a formaldehyde scavenger selected from the group consisting selected from the group consisting of a small molecule scavenger, a polymeric scavenger, a scavenger moiety immobilized on an insoluble polymer support and mixtures thereof;
c) admixing the microcapsules and scavenger;
d) providing a microcapsule product with reduced levels of formaldehyde.

54. The process of claim 53 wherein the adjunct crosslinker represented by the following formula:
(R¹-)mX¹(—R²—Y)n (I)
wherein
X¹ is selected from the group consisting of C, N or NH, Phosphate, aliphatic moiety, aromatic moiety, aliphatic cyclic, partially unsaturated aliphatic cyclic, heteroatom cyclic, carbohydrate;
Y is selected from the group consisting of an amine, amide, carboxyl, enolizable carbonyl, hydroxyl, thiol moieties, and mixture thereof;
R¹ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R² is equal to zero or selected from the group consisting of CH₂, aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers; and
with the proviso in structure (I) n is larger than 1 but can be equal or less than the maximum substitution possible on the X group; m+n is equal to or less than the maximum substitution possible on the X group and wherein the values of m and n are integers or non-integers.

55. The process of claim 53 wherein the adjunct crosslinker is selected from the groups consisting of 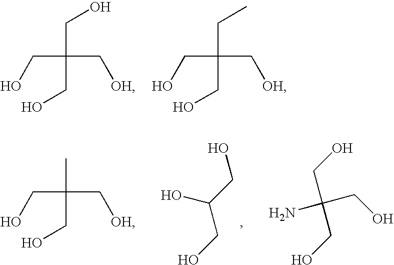
and mixtures thereof.

56. The process of claim 53 wherein the adjunct crosslinker is

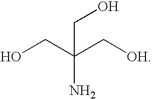

57. The process of claim 53 wherein the adjunct crosslinker is selected from the group consisting of 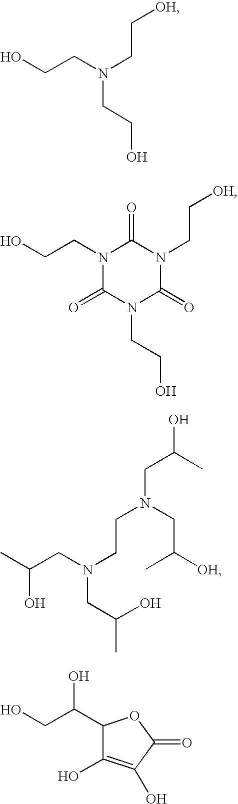
and mixtures thereof.

58. The process of claim 53 wherein the adjunct crosslinker is selected from the group consisting of 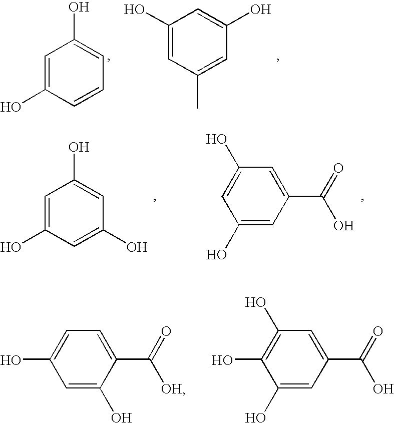
and mixtures thereof.

59. The process of claim 53 wherein the adjunct crosslinker is resorcinol.

60. The process of claim 53 wherein the adjunct crosslinker is represented by the following formula:
(R¹-)m(-R²—Y)nX¹—R³—X²(—R-Z)o(-R⁵)p (II)
wherein
X¹ and X² is independently selected from the group consisting of C, N or NH, Phosphate, aliphatic moiety, aromatic moiety, aliphatic cyclic, partially unsaturated aliphatic cyclic, heteroatom cyclic, carbohydrate;
Y and Z is independently selected from the group consisting of amine, amide, carboxyl, enolizable carbonyl, hydroxyl, thiol moieties, and mixture thereof;
R¹ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R² is equal to zero or selected from the group consisting of, CH₂, an aliphatic moiety, an aromatic moiety, aliphatic cyclic, partially unsaturated aliphatic cyclic, heteroatom cyclic, carbohydrate, polyalkylene oxide, blocked distributions of 2 or more alkylene oxide monomers
R³ is selected from the group consisting of an aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
R⁴ is selected from the group consisting of an aliphatic moiety, an aromatic moiety, aliphatic cyclic, partially unsaturated aliphatic cyclic, heteroatom cyclic, carbohydrate, polyalkylene oxide, blocked distributions of 2 or more alkylene oxide monomers;
R⁵ is equal to zero or selected from the group consisting of CH₂, aliphatic moiety, aromatic moiety, aliphatic cyclic, partially unsaturated aliphatic cyclic, heteroatom cyclic, carbohydrate, polyalkylene oxide, blocked distributions of 2 or more alkylene oxide monomers;
with the proviso in structure (II) the value of n and o are at least 1 but can be equal to or less than the maximum substitution possible on the X¹ and X² groups, respectively, m+n is equal or less than the maximum substitution possible on the X¹ group minus 1, o+p is equal or less than the maximum substitution possible on the X² group minus 1, and wherein the values for m, n, o and p are integers or non-integers.

61. The process of claim 53 wherein the adjunct crosslinker is N,N,N′,N′-tetrakis(2-hydroxypropyl)ethylenediamine.

62. The process of claim 53 where the amount of formaldehyde scavenger is present from an effective trace amount up to about 100 times the molar excess of the molar equivalency of the potential formaldehyde present in the slurry.

63. The process of claim 53 where the amount of formaldehyde scavenger is present from about 0.01 times up to about 10 times the molar excess of the molar equivalency of the potential formaldehyde present in the slurry.

64. The process of claim 53 wherein the levels of formaldehyde are reduced to less than about 1000 ppm.

65. The process of claim 53 wherein the levels of formaldehyde are reduced to less than about 750 ppm.

66. The process of claim 53 wherein the levels of formaldehyde are reduced to less than about 500 ppm.

67. The process of claim 53 wherein the levels of formaldehyde are reduced to less than about 250 ppm.

68. The process of claim 53 wherein the levels of formaldehyde are reduced to less than about 100 ppm.

69. The process of claim 53 wherein the levels of formaldehyde are reduced to less than about 50 ppm.

70. The process of claim 53 wherein the levels of formaldehyde are reduced to less than about 10 ppm.

71. The process of claim 53 wherein the levels of formaldehyde are reduced to less than about 5 ppm.

72. The process of claim 53 wherein the polymeric wall is selected from a vinyl polymer; an acrylate polymer, melamine-formaldehyde; urea formaldehyde and mixtures thereof.

73. The process of claim 72 wherein the mole ratio of melamine-formaldehyde to adjunct crosslinker is in the range of from about 500 to about 0.1.

74. The process of claim 72 wherein the mole ratio of melamine-formaldehyde to adjunct crosslinker is in the range of from about 100 to about 0.5.

75. The process of claim 72 wherein the mole ratio of melamine-formaldehyde to adjunct crosslinker is in the range of from about 50 to about 1.

76. The process of claim 53 wherein the formaldehyde scavenger is a small molecule selected from β-dicarbonyl compounds, amides, imines, acetal formers, sulfur containing compounds, activated carbon, ammonium, organic amines, an oxidizing agent and mixtures thereof.

77. The process of claim 76 wherein the β-dicarbonyl compound is selected from the group consisting of acetoacetamide, ethyl acetoacetate, N,N-Dimethyleneacetamide, acetoacetone, dimethyl-1,3-acetonedicarboxylate, 1,3,-acetonedicarboxylic acid, resorcinol, 1,3-cyclohexadione, barbituric acid, salicyclic acid, 5,5-dimethyl-1,3-cyclohexanedione (dimedone), 2,2-dimethyl-1,3-dioxane-4,6-dione and mixtures thereof.

78. The process of claim 76 wherein the amide compound is selected from the group consisting of urea, ethylene urea, propylene urea, ε-caprolactam, glycouril, hydantoin, 2-oxazolidinone, 2-pyrrolidinone, uracil, barbituric acid, thymine, uric acid, allantoin, 4,5-dihydroxyethylene urea, monomethylol-4-hydroxy-4-methoxy-5,5-dimethyl-propylurea, polyamides, nylon and mixtures thereof.

79. The process of claim 78 wherein the amide compound is ethylene urea.

80. The process of claim 76 wherein the amine compound is selected from the group consisting of poly(vinyl)amine, arginine, lysine, proteins containing lysine and asparagines, hydrazines, aromatic amines, aromatic diamines, aminobenzoic acid derivatives, amine phenols, melamine, 2-amino-2-methyl-1-propanol, benzoguanamine and mixtures thereof.

81. The process of claim 80 wherein the proteins is selected from casein, gelatin, gluten, whey protein, soy protein, collagen and mixtures thereof.

82. The process of claim 80 wherein the hydrazines is 2,4-dinitrophenzylhydrazine.

83. The process of claim 76 wherein the acetal forming compound is selected from the group consisting of diethylene glycol, saccharides, polysaccharides and mixtures thereof.

84. The process of claim 83 wherein the saccharides is selected from glucose, D-sorbitol, sucrose, tannins/tannic acid and mixtures thereof.

85. The process of claim 83 wherein the polysaccharide is a selected from pectin, starch and mixtures thereof.

86. The process of claim 76 wherein the sulfur containing compound is selected from the group consisting of bisulfite, cysteine and mixtures thereof.

87. The process of claim 76 wherein the oxidizing agent is selected from the group consisting of manganese oxide, hydrogen peroxide (H₂O₂), hypochlorite, chlorine, peracids, oxygen, ozone, chlorine dioxygen, sodium percarbonate, sodium perborate and mixture thereof.

88. The process of claim 87 further comprising tetraacetylethylenediamine, transition metal complexes, metalloporphyrins, peroxidases and mixtures thereof.

89. The process of claim 53 wherein the formaldehyde scavenger is polymeric.

90. The process of claim 89 wherein the polymeric scavenger is selected from the group consisting of methacrylic acid, maleic anhydride, maleic acid, itaconic acid, acrylamide, vinyl amine, vinyl alcohol, vinyl mercaptan, saccharides, peptides, allylamin, acrylic acid, olefin, alkylene-oxide, amine, urea, urethane, carbonate, ester, amides, proteins and mixture thereof.

91. The process of claim 90 wherein the end groups of the polymeric scavenger are modified with functional groups selected from the group consisting of β-dicarbonyl compounds, amides, imines, acetal formers, sulfur containing compounds, activated carbon, ammonium, organic amines and mixtures thereof.

92. The process of claim 91 wherein the polymeric scavenger modified with functional end groups is selected from the group consisting of poly(1,4-butanediol)-bis-(4-aminobenzoate) and poly(ethyleneglycol) diacetoacetate.

93. The process of claim 89 wherein the pendant groups of the polymer are modified with functional groups selected from the group consisting of β-dicarbonyl compounds, amides, imines, acetal formers, sulfur containing compounds, activated carbon, ammonium, organic amines and mixtures thereof.

94. The process of claim 53 wherein the solid support is selected from the group consisting of polyolefins such as polyethylene and polystyrene, polyvinylacetate, polysaccharides such as dextran, poly esters, polyamides, polyurethanes, polyacrylates, polyureas, inorganic supports are clays, alumina, silica, zeolite and titanium dioxide.

95. The process of claim 94 wherein the scavenger moiety immobilized on the solid supports is selected from the group consisting of β-dicarbonyl compounds, amides, imines, acetal formers, sulfur containing compounds, activated carbon, ammonium and organic amines.

96. The process of claim 53 wherein the encapsulating polymer is a crosslinked network of polymers comprising a melamine-formaldehyde:acrylamide-acrylic acid copolymer wherein the mole ratio is in the range of from about 9:1 to about 1:9.

97. The process of claim 96 wherein the mole ratio of melamine-formaldehyde:acrylamide-acrylic acid copolymer is in the range of from about 5:1 to about 1:5.

98. The process of claim 96 wherein the mole ratio of melamine-formaldehyde:acrylamide-acrylic acid copolymer is in the range of from about 2:1 to about 1:2.

99. The process of claim 53 wherein the polymeric wall and adjunct crosslinker is cured at a temperature above about 90° C.

100. The process of claim 53 wherein the polymeric wall is cured at a temperature above about 110° C.

101. The process of claim 53 wherein the polymeric wall is cured at a temperature above about 120° C.

102. The process of claim 53 wherein the polymeric wall is cured for up to about one hour.

103. The process of claim 53 wherein the polymeric wall is cured for up to about two hours.

104. The process of claim 53 wherein the polymeric wall is cured for greater than about two hours.

105. The process of claim 53 wherein the pH of the microcapsule product is from about 1 to about 9.

106. The process of claim 53 wherein the pH of the microcapsule product is from about 2 to about 8.

107. The process of claim 53 wherein the pH of the microcapsule product is from about 3 to about 6.

108. The process of claim 53 wherein the microcapsule product is further coated by a cationic polymer.

109. The process of claim 108 wherein the cationic polymer is selected from polysaccharides, cationically modified starch and cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides, imidazolium halides and mixtures.

110. The method of claim 109 wherein the cationic polymer is selected from a cationically modified starch, cationically modified guar and mixtures thereof.

111. A method of imparting an olfactory effective amount of a fragrance into a consumer product comprising incorporating at least about 0.25 weight % of the capsules of produced according to the process of claim 53 into a consumer product.

112. The method of claim 111 wherein the consumer product is selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, automatic dishwashing compositions, foodstuffs, beverages and mixtures thereof.

113. A microcapsule product produced according to the process of claim 53.

114. A consumer product selected from the group consisting of laundry detergent, fabric softeners, bleach products, tumble dryer sheets, liquid dish detergents, automatic dish detergents, hair shampoos, hair conditioners, toothpastes, mouthwash, oral care products, liquid soaps, body wash, lotions, creams, hair gels, anti-perspirants, deodorants, shaving products, colognes, bodywash, automatic dishwashing compositions, foodstuffs, beverages and mixtures thereof comprising the microcapsule product according to the process of claim 53.

115. The consumer product of claim 114 further comprising about 0.01 times up to about 100 times the molar amount of all the formaldehyde in the consumer product of formaldehyde scavenger selected from the group consisting of β-dicarbonyl compounds, amides, imines, acetal formers, sulfur containing compounds, activated carbon, ammonium, organic amines, an oxidizing agent, a polymeric scavenger, a scavenger moiety immobilized on an insoluble polymer support and mixtures thereof.
